(19) United States
(12) Patent Application Publication
(10) Pub. No.: US 2017/0065288 A1
(43) Pub. Date: Mar. 9, 2017

(54) MEDICAL DEVICE AND TREATMENT METHOD

(71) Applicant: TERUMO KABUSHIKI KAISHA, Tokyo (JP)

(72) Inventors: Masaomi Imai, Ashigarakami-gun (JP); Yuuki Masubuchi, Ashigarakami-gun (JP); Takashi Kitaoka, Ashigarakami-gun (JP); Takahiro Chida, Ashigarakami-gun (JP); Kazuaki Kanamoto, Ashigarakami-gun (JP)

(73) Assignee: TERUMO KABUSHIKI KAISHA, Tokyo (JP)

(21) Appl. No.: 15/255,456

(22) Filed: Sep. 2, 2016

(30) Foreign Application Priority Data
Sep. 7, 2015 (JP) 2015-0175358

(51) Int. Cl. A61B 17/22; A61B 17/3207

(52) U.S. Cl. CPC A61B 17/22; A61B 17/320725; A61B 2017/00862

(57) ABSTRACT

A medical device is disclosed for restricting a flow in a body lumen by being inserted into the body lumen. The medical device including an elongated shaft part; an expansion part which is a tubular body having a plurality of gaps and elastically deformable, with the shaft part being connected to at least one of both side end portions of the tubular body; a cover part which is flexibly deformable independently from the expansion part while surrounding an outer circumference of the expansion part, which is tubular in shape, both end portions of the tubular shape being connected to both end portions of the expansion part; and a sheath in which the expansion part and the cover part both contracted in diameter can be accommodated.

### CROSS-REFERENCES TO RELATED APPLICATIONS

This application claims priority to Japanese Application No. 2015-0175358 filed on Sep. 7, 2015, the entire content of which is incorporated herein by reference.

### TECHNICAL FIELD

The present disclosure relates to a medical device and a treatment method which are to be used for removing a substance present in a body lumen.

### BACKGROUND DISCUSSION

For example, when part of a vein is clogged with a thrombus, a pain and/or swelling may be generated. For treatment of this symptom, a method of percutaneously inserting a thrombus removal device and thereby removing the thrombus may be used. In such a treatment, if the thrombus peeled completely or partly from the blood vessel wall is conveyed by the blood flow to reach the lung, there arises a danger of pulmonary thrombosis. In performing such a treatment, therefore, a thrombolytic agent may be used before or after the treatment and/or during the treatment, or the peeled thrombus may be sucked and removed as assuredly as possible during the treatment. Even when such an auxiliary treatment is conducted, however, a peeled thrombus having such a size as to cause a problem clinically may reach the lung.

As a method for obviating such pulmonary thrombosis, the use of an inferior vena cava filter (IVC filter) for capturing the thrombus flowing in a blood vessel has been known (see, for example, U.S. Pat. No. 8,182,507).

Since the gaps (openings) in the IVC filter are wide, however, only large thrombi can be captured. In addition, the IVC filter is designed for the inferior vena cava, and is therefore not suited to blood vessels smaller in diameter than the inferior vena cava. In addition, in the case of sucking the thrombus captured by the filter, suction against a strong blood flow must be performed, which is very difficult to achieve successfully.

### SUMMARY

Thus, there is a need for a medical device and a treatment method which can be applied to body lumens of a wide range of inside diameter and by which it is possible to restrict a flow in a body lumen and to effectively remove a substance present in the body lumen.

A medical device is disclosed for restricting a flow in a body lumen by being inserted into the body lumen. The medical device can include an elongated shaft part; an expansion part which is a tubular body having a plurality of gaps and elastically deformable, an outside diameter of the tubular body being greater at a central portion than at both side end portions of the tubular body in a natural state where no external force is acting, with the shaft part being connected to at least one of the both side end portions of the tubular body; a cover part which is flexibly deformable independently from the expansion part while surrounding an outer circumference of the expansion part, which is tubular in shape, both end portions of the tubular shape being connected to both end portions of the expansion part, and which is capable of forming an overlapping part overlapping by being folded back in an axial direction such that inner surface portions of the tubular shape contact each other when the expansion part is expanded; and a sheath in which the expansion part and the cover part both contracted in diameter can be accommodated.

In the medical device configured as above-mentioned, with the expansion part and the cover part released from the sheath, the expansion part expands in accordance with the shape of a body lumen by its own elastic force, an overlapping part projecting in the radial direction by overlapping is formed in the cover part along the whole circumference of the cover part, and, concurrently, the cover part is pressed against the body lumen by the expansion part. Therefore, the range of inside diameter of the body lumen to which the medical device is applicable is widened by the expansion part which expands by its own elastic force. In addition, a flow in the body lumen can be effectively restricted by the cover part formed with the overlapping part, and the substance can be effectively removed from inside the body lumen.

A medical device is disclosed for restricting a flow in a body lumen by being inserted into the body lumen, the medical device comprising: an elongated shaft; an expandable tubular body having a plurality of gaps and elastically deformable, the expandable tubular body having a central portion, a tapered proximal-side portion, and a taper distal-side portion, and the elongated shaft being connected to at least one end portion of the expandable tubular body; and a tubular cover which is flexibly deformable independently from the expandable tubular body while surrounding an outer circumference of the expandable tubular body, both end portions of the tubular cover being connected to both end portions of the expandable tubular body, and which is capable of forming an overlapping part overlapping by being folded back in an axial direction such that inner surface portions of the tubular cover contact each other when the expandable tubular body is expanded.

### BRIEF DESCRIPTION OF THE DRAWINGS

FIG. 1 is a plan view of a medical device according to an embodiment of the present disclosure;

FIG. 2 is a plan view illustrating a restriction instrument, a pushing shaft, and a sheath of the medical device according to the embodiment in an assembled state;

FIGS. 3A and 3B are plan views of a cover part of the restriction instrument, wherein FIG. 3A illustrates the cover part in an expanded state, and FIG. 3B illustrates the cover part in a contracted state;

FIGS. 4A and 4B are perspective views of an expansion part in the cover part of the restriction instrument, wherein FIG. 4A illustrates the expansion part in an expanded state, and FIG. 4B illustrates the expansion part in a contracted state;

FIG. 5 is an enlarged sectional view of a proximal-side connection part and a distal-side connection part;

FIG. 6 is a plan view illustrating the expansion part of the restriction instrument in a contracted state;

FIG. 7 is a sectional view taken along line VII-VII of FIG. 5;

FIG. 8 is a plan view of a removal device;

FIG. 9 is a perspective view of a distal portion of the removal device;

FIG. 10 is a sectional view of the distal portion of the removal device;

FIGS. 11A and 11B are sectional views illustrating a state inside a blood vessel, wherein FIG. 11A illustrates a state where the medical device is inserted in the blood vessel, and FIG. 11B illustrates a state where distal portions of the expansion part and the cover part are partially expanded within the blood vessel;

FIGS. 12A and 12B are sectional views illustrating a state inside a blood vessel, wherein FIG. 12A illustrates a state when an overlapping part is formed, and FIG. 12B illustrates a state after the overlapping part is formed;

FIG. 13 is a sectional view taken along line XIII-XIII of FIG. 12A;

FIG. 14 is a sectional view taken along line XIV-XIV of FIG. 13;

FIGS. 15A and 15B are sectional views illustrating a state inside a blood vessel, wherein FIG. 15A illustrates a state in which the expansion part and the cover part are entirely released from the sheath, and FIG. 15B illustrates a state in which the restriction instrument is set remaining inside the blood vessel;

FIGS. 16A and 16B are sectional views illustrating a state inside a blood vessel, wherein FIG. 16A illustrates a state in which the removal device is inserted in the blood vessel, and FIG. 16B illustrates a state in which a stirring portion of the removal device is expanded;

FIG. 17 is a sectional view illustrating a state inside a blood vessel when the stirring portion of the removal instrument is expanded;

FIG. 18 is an enlarged sectional view of a distal portion of the removal device, illustrating a state in which crushed thrombus is sucked into an opening of an outer tube;

FIG. 19 is an enlarged sectional view of the distal portion of the removal device, illustrating a process in which the thrombus sucked into the opening of the outer tube is cut off by an inner tube;

FIG. 20 is an enlarged sectional view of a distal portion of the removal device, illustrating a state in which the thrombus cut off by the inner tube is cut by a cutting unit;

FIG. 21 is an enlarged sectional view of the distal portion of the removal device illustrating a process in which the thrombus cut by the cutting unit is sucked toward the proximal side of the inner tube;

FIGS. 22A and 22B are sectional views illustrating a state inside a blood vessel, wherein FIG. 22A illustrates a state in which the thrombus adhering to the restriction instrument is being sucked, and FIG. 22B illustrates a state in which the stirring portion is accommodated in an outermost sheath body;

FIGS. 23A and 23B are sectional views illustrating a state inside a blood vessel, wherein FIG. 23A illustrates a state in which the removal device is drawn out of the blood vessel, and FIG. 23B illustrates a state in which the cover part is accommodated in the sheath; and

FIG. 24 is a plan view of a modification of the expansion part and the cover part of the restriction instrument.

### DETAILED DESCRIPTION

Embodiment of the present disclosure will be described below referring to the drawings. Note that size ratios in the drawings may be exaggerated and be different from the actual ratios, for the convenience of illustration.

A medical device 10 according to an embodiment of the present disclosure is to be used for restraining a flow inside a blood vessel in order to suck and remove a body such as a thrombus or plaque present within the blood vessel. Note that herein the side on which a device is inserted into a blood vessel will be referred to as “distal side” whereas the side of an operator's hand operation will be referred to as “proximal side.” In addition, the body to be removed is not necessarily limited to a thrombus or plaque but may be any substance that can exist in a body lumen.

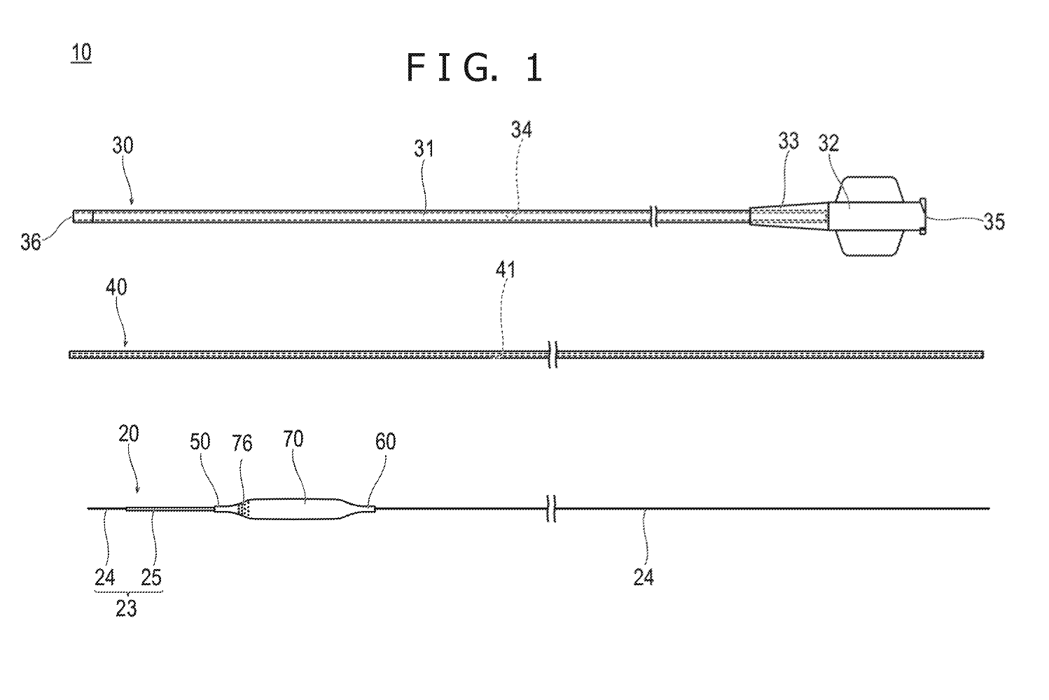

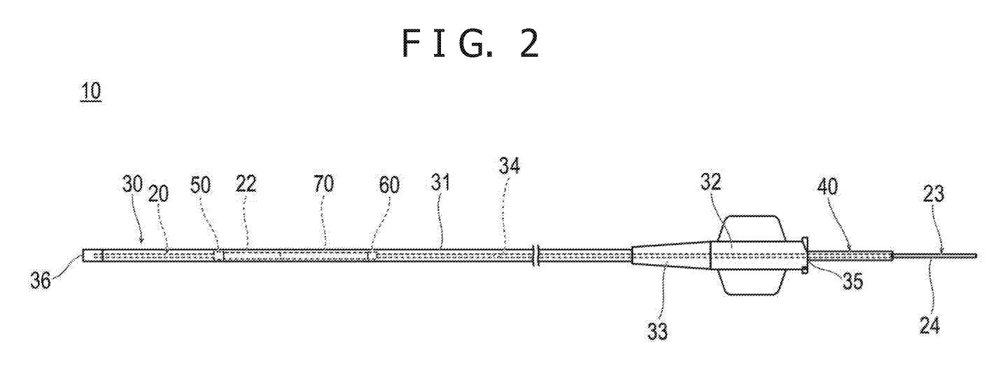

As illustrated in FIGS. 1 and 2, the medical device 10 according to the embodiment of the present disclosure can include a restriction instrument 20 for restricting a flow of blood inside a blood vessel, a sheath 30 capable of accommodating the restriction instrument 20 therein, and a pushing shaft 40 used for pushing out the restriction instrument 20 from the sheath 30.

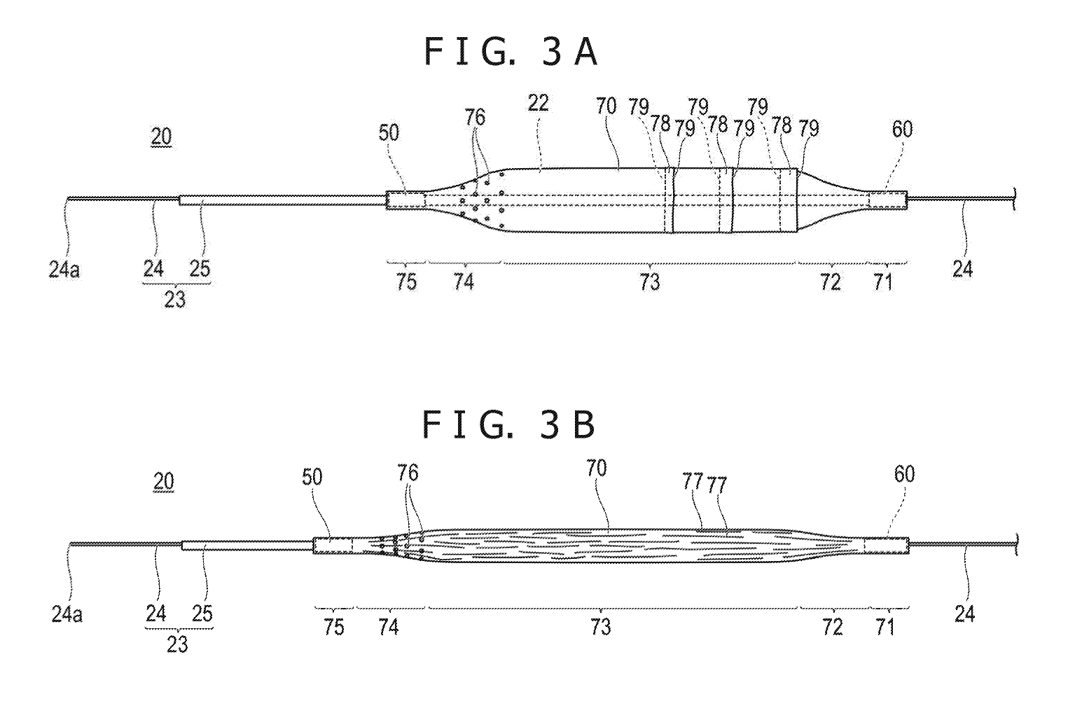

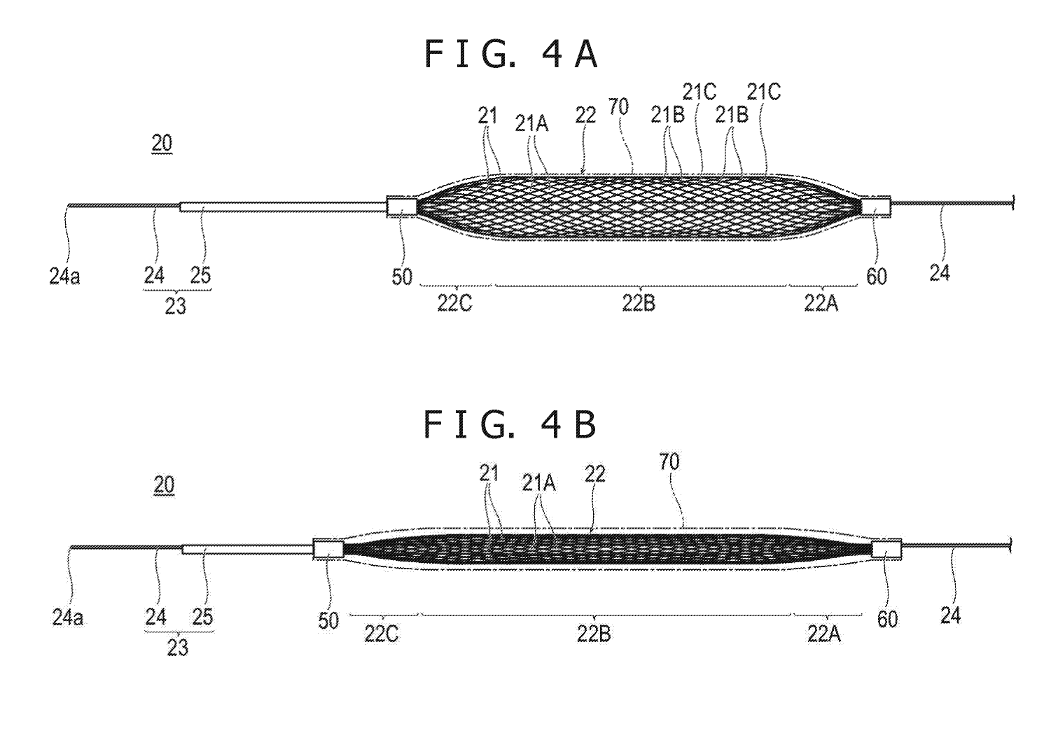

As illustrated in FIGS. 3A to 4B, the restriction instrument 20 can include an expansion part 22 which is a net-formed tubular body provided with a plurality of gaps 21A and elastically deformable, a cover part 70 surrounding an outer circumference of the expansion part 22, and an elongated shaft part 23 penetrating the expansion part 22 and the cover part 70.

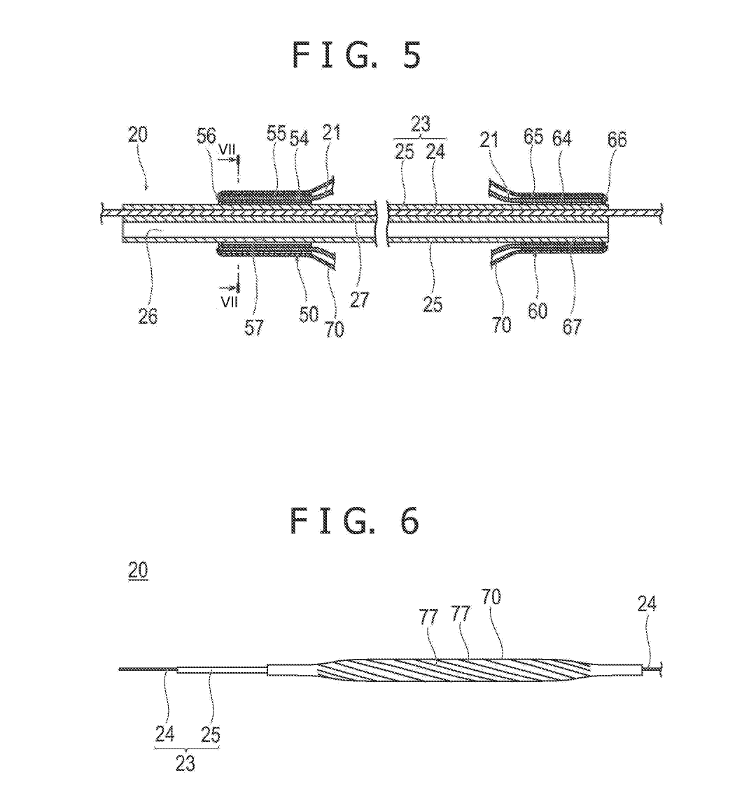

As illustrated in FIGS. 1 to 6, the shaft part 23 can include an elongated wire part 24, and a guide wire tubular body 25 which is fixed to a distal portion of the wire part 24 and which is formed therein with a guide wire lumen 26. To the guide wire tubular body 25, either an inner circumferential face 57 of an inner tube 54 provided at a distal portion of the expansion part 22 or an inner circumferential face 67 of an inner tube 64 provided at a proximal portion of the expansion part 22 (in this embodiment, the inner circumferential face 67) is fixed. The guide wire tubular body 25 is formed therein with a wire through-hole 27 in which the wire part 24 is inserted and fixed, the wire through-hole 27 being parallel to the guide wire lumen 26. Note that a tip 24a of the wire part 24 may be fixed to the inner circumferential face 67 of the inner tube 64 at a proximal portion of the expansion part 22. In this instance, the wire part 24 and the guide wire tubular body 25 of the shaft part 23 are separate bodies.

The constituent material of the wire part 24 constituting the shaft part 23 is not particularly limited; for example, stainless steel, and shape memory alloys can be used preferably. The constituent material of the guide wire tubular body 25 constituting the shaft part 23 is not particularly restricted; for example, plastic materials such as polyimides and polyamides as well as stainless steel, and shape memory alloys can be used preferably.

As depicted in FIGS. 4A and 4B, the expansion part 22 can include a plurality of wire-like members (or wires) 21 which are knitted in a net form so as to constitute a tubular body having gaps and which are flexibly deformable, and a distal-side connection part 50 and a proximal-side connection part 60 which are connected to the guide wire tubular body 25 of the shaft part 23. At either of the inner circumferential faces 57 and 67 of the inner tubes 54 and 64 of the distal-side connection part 50 and the proximal-side connection part 60 (in this embodiment, the inner circumferential face 67), an outer circumferential face of the guide wire tubular body 25 of the shaft part 23 is fixed. At the other of the inner circumferential faces 57 and 67 (in this embodiment, the inner circumferential face 57), the outer circumferential face of the guide wire tubular body 25 is not fixed but is disposed to be slidable. The expansion part 22 is formed in a tubular shape such as to have gaps 21A between the wire-like members 21, by knitting the plurality of wire-like members 21.

The expansion part 22 can be deformed into an expanded state of being expanded in outside diameter by an elastic force (restoring force) of the wire-like members 21 themselves in a condition where no external force is exerted thereon as illustrated in FIG. 4A, and a contracted state of being reduced in diameter through elastic deformation as illustrated in FIG. 4B. The expansion part 22 can include a proximal-side tapered portion 22A of the expansion part 22 where inside and outside diameters are gradually increased from an expansion part proximal portion toward the distal side, an expansion part central portion 22B which is located on the distal side of the proximal-side tapered portion 22A of the expansion part 22 and at which the outside diameter is substantially constant, and a distal-side tapered portion 22C of the expansion part 22 where the inside and outside diameters are gradually decreased from the expansion part central portion 22B toward the distal side. The expansion part central portion 22B is a portion, which by expanding, presses the cover part 70 against an inner wall of a blood vessel. Note that the expansion part central portion 22B, which by expanding, presses the cover part 70 against the blood vessel inner wall may be provided in the form of being divided into a plurality of portions along an axial direction. A maximum outside diameter of the expansion part 22 when expanded by its own expanding force before being covered by the cover part 70 is greater than a maximum outside diameter of the cover part 70.

As illustrated in FIGS. 3A to 5, the proximal-side connection part 60 can include the inner tube 64 located inside the wire-like members 21, an outer tube 65 located outside the wire-like members 21, and a connection part 66 connecting the inner tube 64 and the outer tube 65 at an end portion, with the wire-like members 21 being clamped and fixed between the inner tube 64 and the outer tube 65. At the proximal-side connection part 60, the inner tube 64 is firmly attached to the guide wire tubular body 25. Note that the connection part 66 may not necessarily be provided, so long as the wire-like members 21 can be fixed.

The distal-side connection part 50 can include the inner tube 54 located inside the wire-like members 21, an outer tube 55 located outside the wire-like members 21, and a connection part 56 connecting the inner tube 54 and the outer tube 55 at an end portion, with the wire-like members 21 clamped and fixed between the inner tube 54 and the outer tube 55. At the distal-side connection part 50, the guide wire tubular body 25 is slidably inserted inside the inner tube 54, whereby a gap is formed between the inner tube 54 and the guide wire tubular body 25, and the distal-side connection part 50 can be moved in an axial direction relative to the guide wire tubular body 25. Note that the connection part 56 may not necessarily be provided, so long as the wire-like members 21 can be fixed. The gap between the inner tube 54 and the guide wire tubular body 25 is preferably 0.01 mm to 1.0 mm.

With the expansion part 22 put into an expanded state, the distal-side connection part 50 is slid proximally relative to the guide wire tubular body 25 to approach the proximal-side connection part 60 (see FIGS. 3A and 4A), and, with the expansion part 22 put into a contracted state, the distal-side connection part 50 is slid distally relative to the guide wire tubular body 25 to move away from the proximal-side connection part 60 (see FIGS. 3B and 4B). The distal-side connection part 50 can be moved closer to or away from the proximal-side connection part 60, whereby the outside diameter of the knitted expansion part 22 can be greatly changed.

The number of the wire-like members 21 is not particularly limited, and may be, for example, four to 72. In addition, the conditions for knitting of the wire-like members 21 are not particularly limited.

The outside diameter of the wire-like members 21 can be appropriately selected according to the material of the wire-like members 21 and the use of the expansion part 22, and may be, for example, 20 μm to 300 μm.

The wire-like members 21 preferably include wire-like members 21B and wire-like members 21C which differ in outside diameter. The wire-like members 21B are greater than the wire-like members 21C in outside diameter. The outside diameter of the wire-like members 21B is, for example, 200 μm, and the outside diameter of the wire-like members 21C is, for example, 120 μm. In this embodiment, two wire-like members 21B and one wire-like member 21C are alternately arranged, and 16 wire-like members 21B and eight wire-like members 21C are used. By the use of the wire-like members 21B and 21C differing in outside diameter for forming the expansion part 22, it can help ensure that when the expansion part 22 is contracted and accommodated into the sheath 30, the thinner wire-like members 21C are not liable to make contact with the inner wall surface of the sheath 30 through the cover part 70, so that the positions of intersections of the meshing are not easily deviated, and the shape of the expansion part 22 is stabilized. Where the number of the thicker wire-like members 21B is greater than the number of the thinner wire-like members 21C, an expanding force of the expansion part 22 can be kept great, and the shape is stabilized. Note that the number of the thicker wire-like members may be smaller than or equal to the number of the thinner wire-like members. Where the number of the thicker wire-like members is smaller than the number of the thinner wire-like members, the expansion part is flexible, and can easily follow up to the shape of a body lumen.

The constituent material of the wire-like members 21 is preferably a flexible material. Examples of the material which can be preferably used for constituting the wire-like members 21 include shape memory alloys to which a shape memory effect or hyperelasticity is imparted by a heat treatment, stainless steel, tantalum (Ta), titanium (Ti), platinum (Pt), gold (Au), tungsten (W), polyolefin such as polyethylene, polypropylene, etc., polyamides, polyesters such as polyethylene terephthalate, etc., fluorine-containing polymers such as ETFE (tetrafluoroethylene-ethylene copolymer), etc., PEEK (polyether ether ketone), polyimides, etc. As the shape memory alloys, there can be preferably used Ni—Ti-based, Cu—Al—Ni-based, and Cu—Zn—Al-based alloys, and combinations of them. Examples of a structure in which a plurality of materials are combined include a structure wherein a Pt core wire is coated with a Ni—Ti alloy and a structure wherein a Ni—Ti alloy core wire is plated with gold, for imparting contrast properties.

Outside diameters of the outer tubes 55 and 65 are not particularly limited, and can be, for example, 0.3 mm to 3.0 mm. Inside diameters of the inner tubes 54 and 64 are not specifically restricted, and can be, for example, 0.1 mm to 2.0 mm.

The constituent material or materials of the inner tubes 54 and 64 and the outer tubes 55 and 65 are not particularly limited; for example, stainless steel, and shape memory alloys can be used.

The maximum outside diameter of the expansion part 22 can be appropriately selected according to the inside diameter of a blood vessel to which the medical device 10 is applied, and the maximum outside diameter can be, for example, 1 mm to 40 mm. The outside diameter of the expansion part 22 in a contracted state can be appropriately selected according to the inside diameter of a blood vessel to which the medical device 10 is applied, and the outside diameter can be, for example, 0.3 mm to 4.0 mm. The axial length of the expansion part 22 in the contracted state can be appropriately selected according to a blood vessel to which the medical device 10 is applied, and the axial length can be, for example, 20 mm to 150 mm.

As illustrated in FIGS. 3A and 3B, the cover part 70 is a member formed in a tubular form from a thin film in such a manner as to cover the outer periphery of the whole body of the expansion part 22.

The cover part 70 can include a cover proximal portion 71 firmly attached to an outer peripheral surface of the proximal-side connection part 60, and a cover distal portion 75 firmly attached to an outer circumferential face of the distal-side connection part 50. The cover part 70 further can include a proximal-side tapered portion 72 where inside and outside diameters gradually increase from the cover proximal portion 71 toward the distal side, a cover central portion 73 which is located on the distal side of the proximal-side tapered portion 72 and at which the outside diameter is substantially constant, and a distal-side tapered portion 74 where inside and outside diameters gradually decrease from the cover central portion 73 toward the distal side. The cover part 70 can be firmly attached to the expansion part 22 only at the cover proximal portion 71 and the cover distal portion 75; thus, the proximal-side tapered portion 72, the cover central portion 73, and the distal-side tapered portion 74 are not firmly attached to the expansion part 22 but only cover the expansion part 22. Therefore, the cover part 70 can be deformed independently from the expansion part 22, and can be separated from the expansion part 22 so as not to contact the expansion part 22, at all its portions exclusive of both end portions. Accordingly, the positions where the expansion part 22 and the cover part 70 contact each other when expanded are different from those when contracted. In addition, since the cover part 70 can be deformed independently from the expansion part 22, the intersection angle of the wire-like members 21 constituting the expansion part 22 can vary without being hindered by the cover part 70, so that the expansion part 22 can be deformed flexibly. In addition, the expansion part 22 becomes smaller in axial length when enlarged in diameter, since its outside diameter varies while the intersection angle of the wire-like members 21 varies concurrently. By contrast, the cover part 70 is formed of a high-strength material such as not to be broken even though it is thin, and the axial length of the cover part 70 does not vary so much as the axial length of the expansion part 22 does.

As illustrated in FIG. 3B, when the cover part 70 is contracted, the cover part 70 is contracted in diameter such that folded-back parts 77 folded back in an overlapping manner are generated, and edge portions of the folded-back parts 77 formed in a winkle-like manner extend in the axial direction. In accordance with an exemplary embodiment, It can be preferable that the folded-back parts 77 are formed in plurality in the circumferential direction, they are not formed over the whole axial length of the cover part 70 but are intermittently formed to be shorter than the whole axial length of the cover part 70, and are formed in plurality in the axial direction. Note that each of the folded-back parts 77 may be formed over the whole axial length of the cover part 77. Since the cover part 70 is smaller than the expansion part 22 in variation in axial length, the axial length of the cover part 70 is set to be equal to or slightly larger than the axial length of the expansion part 22 in a contracted state in which the axial length of the expansion part 22 is enlarged. In this state, overlapping parts 78 (see FIG. 3A) which overlap in the manner of folding back in the axial direction are not formed in the cover part 70.

In addition, as depicted in FIG. 3A, when the cover part 70 is expanded, the cover part 70 is expanded in diameter in such a manner that the folded-back parts 77 are stretched and the number or amount of folded parts is reduced. For example, the cover part 70 has a structure such that its outside diameter varies as the folded-back parts 77 folded back in the manner that the inner circumferential face portions contact each other are formed or as the folded-back parts 77 are stretched. Note that when the cover part 70 is expanded, the folded-back parts 77 may not be stretched completely and folded parts may be left partially. In the expanded state, since the axial length of the expansion part 22 is shortened, at least one overlapping part 78 folded to overlap in the axial direction can be formed in the cover part 70, by utilizing a surplus axial length of the cover part 70 in the expanded state. In addition, a configuration may be adopted in which when the blood vessel diameter is smaller than the maximum diameter of the expansion part 22 in a natural state, the overlapping parts 78 are formed, a one-side surface of each overlapping part 78 folded back makes contact with the blood vessel inner wall, whereas the other-side surface of the overlapping part 78 makes contact with the outer surface of the cover part 70. The cover part 70 may have preliminary shape parts 79 pre-shaped by heating in a folded and overlapping state, at positions where the overlapping parts 78 are to be formed.

The distal-side tapered portion 74 of the cover part 70 is formed with at least one hole portion 76. The hole portion 76 functions to let blood flow into the inside when the cover part 70 expands with an increase in inside volume, and functions to discharge the blood to the outside when the cover part 70 contracts with a decrease in the inside volume. With the hole portion or portions 76 formed in the distal-side tapered portion 74, the hole portions 76 are not shut up even when the cover central portion 73 makes contact with the inner wall surface of a blood vessel, so that blood can flow through the hole portions 76 favorably. The diameter of the hole portions 76 is not particularly limited, and can be, for example, 0.1 mm to 2 mm.

The cover part 70 functions to restrict a blood flow in such a manner that a thrombus in a blood vessel can be effectively sucked and removed by a removal device 100 which will be described later. Therefore, it can be desirable that the cover part 70 is not provided with a hole portion or portions on the proximal side and does not permit blood to flow from the proximal side to the distal side of the cover part 70.

The maximum inside diameter of the cover part 70 is smaller than the maximum outside diameter of the expansion part 22 in the state of being expanded without being covered with the cover part 70. For example, the cover part 70 is forcibly restraining an expansion in diameter of the expansion part 22, by surrounding the expansion part 22. Accordingly, even when the cover part 70 is in an expanded state, the expanding force of the expansion part 22 can be effectively exhibited. The maximum outside diameter of the cover central portion 73 of the cover part 70 in the expanded state is greater than the inside diameter of a blood vessel to which the medical device 10 is applied, such that the cover central portion 73 can contact the inner wall surface of the blood vessel to which the medical device 10 is applied.

In accordance with an exemplary embodiment, as illustrated in FIG. 6, the cover part 70 may be provided in the circumferential direction with a plurality of folded-back parts 77 inclined relative to the axial direction, namely, folded-back parts 77 set at an angle relative to the axial direction, before insertion into the sheath 30. This can help ensure that when the cover part 70 is inserted into the sheath 30 or discharged from the sheath 30, it is easy to perform the insertion and discharge with twisting, so that the resistance at the time of inserting or discharging the cover part 70 can be reduced. Note that the cover part 70 may be formed of a highly stretchable material so that the cover part 70 can be expanded and contracted in diameter without generating the folded-back parts.

The maximum outside diameter of the cover central portion 73 of the cover part 70 in an expanded state is greater than the inside diameter of the blood vessel to which the medical device 10 is applied, and the maximum outside diameter in the expanded state can be appropriately selected according to the blood vessel to which the medical device 10 is applied. This maximum outside diameter in the expanded state can be, for example, 1 mm to 40 mm. The maximum outside diameter of the cover part 70 in a contracted state is smaller than the inside diameter of a blood vessel to which the medical device 10 is applied, and the maximum outside diameter in the contracted state can be appropriately selected according to the blood vessel to which the medical device 10 is applied. This maximum outside diameter in the contracted state can be, for example, 0.3 mm to 4.0 mm. The axial length of the expansion part 22 in a contracted state can be appropriately selected according to a blood vessel to which the medical device 10 is applied, and the axial length in the contracted state can be, for example, 20 mm to 150 mm.

Note that if the outside diameter of the cover part 70 is too large, when the cover part 70 is accommodated in the sheath 30 the accommodating space inside the sheath 30 would be insufficient; therefore, the resistance at the time of accommodating the cover part 70 into the sheath 30 and the resistance at the time of discharging the cover part 70 from the sheath 30 would be large. Accordingly, it is preferable that the outside diameter of the cover part 70 has a required minimum value.

In addition, if the cover part 70 is too long in the axial direction, when the cover part 70 is accommodated in the sheath 30 the accommodating space inside the sheath 30 would be insufficient; therefore, the resistance at the time of accommodating the cover part 70 into the sheath 30 and the resistance at the time of discharging the cover part 70 from the sheath 30 would be large. Accordingly, it is preferable that the length of the cover part 70 has a required minimum value.

The constituent material of the cover part 70 is preferably a material which is thin, has such strength as not to be broken when deformed, and has such a small frictional resistance as to be slidable within the sheath 30. For example, polyethylene can be applied as the material. The thickness of the cover part 70 is not particularly limited, and can be, for example, 5 μm to 30 μm. The cover part 70 has such an axial length in an expanded state as to completely cover the expansion part 22. Note that the cover part 70 may not necessarily cover the expansion part 22 completely, but may cover only part of the expansion part 22.

In accordance with an exemplary embodiment, the cover part 70 may not be a film-shaped member. The cover part 70 may, for example, be a mesh-shaped film body or be a knitted body knitted from a wire-like member.

An inside surface of the cover part 70 may be coated with a silicone resin, for example, a fluorine-containing resin such as Teflon (registered trademark), or a hydrophilic polymer, in order to enhance slidability. Examples of the hydrophilic polymer can include polyhydroxyethyl methacrylate, polyhydroxyethyl acrylate, hydroxypropyl cellulose, methyl vinyl ether-maleic anhydride copolymer, polyethylene glycol, polyacrylamide, and polyvinylpyrrolidone. With the slidability of the inside surface of the cover part 70 enhanced, it becomes relatively easy to retract the cover part 70 into the sheath 30 after expanding the cover part 70 inside a blood vessel. Note that the treatment for enhancing the slidability may be applied not only to an inner circumferential face of the cover part 70 but also to an outer circumferential face of the cover part 70 in an range exclusive of the cover central portion 73 where a contacting force for secure contact with the blood vessel wall is needed. In this case, it becomes easier to retract the cover part 70 into the sheath 30 after expanding the cover part 70 inside a blood vessel.

As illustrated in FIGS. 1 and 2, the sheath 30 can include a sheath tubular body 31, a hub 32, and an anti-kinking protector 33. The sheath tubular body 31 is provided with a lumen 34 in which the restriction instrument 20 can be accommodated, and is opening at a tubular body opening 36 formed at a distal-side end portion. The hub 32 is fixed to a proximal-side end portion of the sheath tubular body 31, and is provided with a hub opening 35, which communicates with the lumen 34. The anti-kinking protector 33 is a flexible member covering a connection part of the sheath tubular body 31 and the hub 32, and can help inhibit the sheath tubular body 31 from kinking.

The constituent material of the sheath tubular body 31 is not particularly restricted. Examples of the material which can be preferably used here can include polyolefin such as polyethylene, polypropylene, ethylene-propylene copolymer, ethylene-vinyl acetate copolymer, polyvinyl chloride, polystyrene, polyamides, polyimides, and combinations thereof. The sheath tubular body 31 may be composed of a plurality of materials, and a reinforcing member such as a wire-like member may be embedded in the sheath tubular body 31.

The pushing shaft 40 is a tubular body capable of being accommodated in the lumen 34 of the sheath 30, and is formed therein with a pushing-out lumen 41 in which the wire part 24 of the restriction instrument 20 can be inserted. The inside diameter of the pushing-out lumen 41 is smaller than the outside diameter of the proximal-side connection part 60 of the restriction instrument 20. Therefore, the proximal-side connection part 60 cannot enter into the pushing-out lumen 41, and, accordingly, the proximal-side connection part 60 can be pushed in the distal direction by the pushing shaft 40.

Now, the removal device 100 which is inserted into a blood vessel and used for removing a thrombus will be described below.

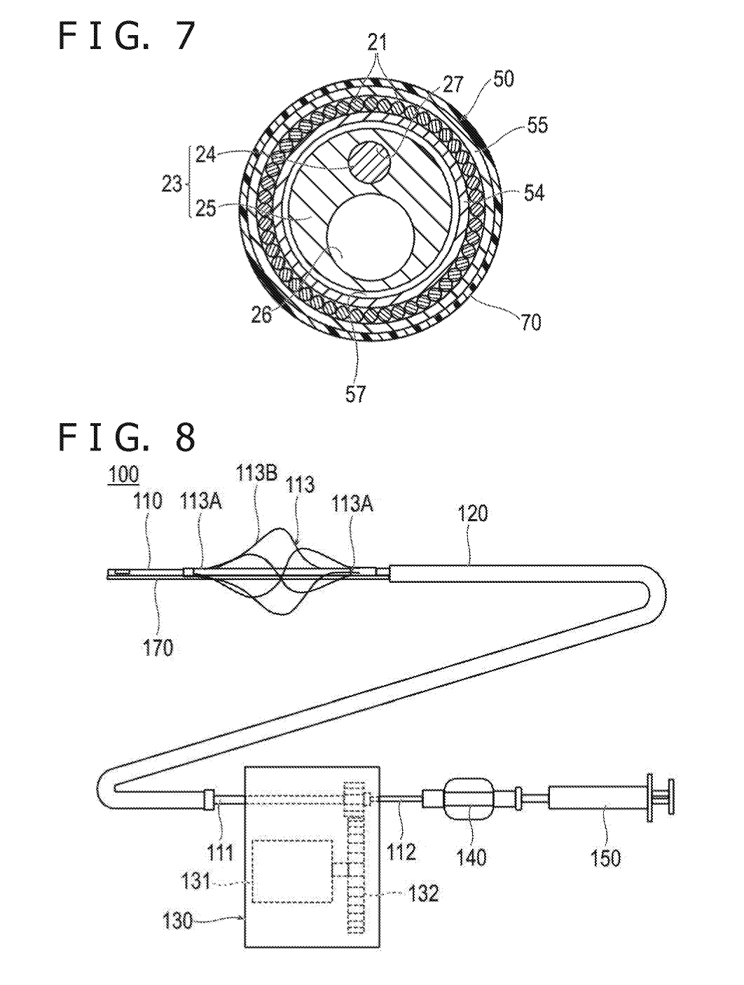

As illustrated in FIGS. 8 to 10, the removal device 100 can include a shaft main body 110 formed in an elongated shape, an outermost sheath body 120 in which the shaft main body 110 is contained and which is axially slidable relative to the shaft main body 110, and a guide wire tubular body 170 formed therein with a second guide wire lumen 171. The removal device 100 further can include a rotationally driving unit 130 capable of rotating the shaft main body 110, a hub 140 provided at a proximal-side end portion of the shaft main body 110, and a syringe 150 connected to the proximal side of the hub 140.

The shaft main body 110 can include a shaft outer tube 111 and a shaft inner tube 112 which are each formed in an elongated hollow shape. The shaft outer tube 111 and the shaft inner tube 112 are each provided therein with a lumen. The inside diameter of the shaft outer tube 111 is greater than the outside diameter of the shaft inner tube 112, and the shaft inner tube 112 is contained in the hollow inside of the shaft outer tube 111. In addition, the shaft inner tube 112 is axially slidable relative to the shaft outer tube 111.

The shaft outer tube 111 has its distal-side end portion forming a distal portion of the shaft main body 110, and has its proximal-side end portion located at the rotationally driving unit 130. The shaft inner tube 112 has its proximal-side end portion extending to the proximal side beyond the proximal-side end portion of the shaft outer tube 111, and connected to the hub 140. By the syringe 150 connected to the hub 140, the hollow inside of the shaft inner tube 112 can be sucked to establish a negative pressure state.

The guide wire tubular body 170 is disposed in the state of being firmly attached to the shaft outer tube 111 along the shaft outer tube 111. The guide wire tubular body 170 is formed with the second guide wire lumen 171 in which a guide wire can be inserted.

The shaft outer tubular 111 is formed of a material which is flexible and has such a characteristic property as to be able to distally transmit rotational power acting from the proximal side. The shaft inner tube 112 is formed of a material, which is flexible and has such a characteristic property as to be able to distally transmit forward and backward reciprocating power acting from the proximal side. For example, the shaft outer tube 111 and the shaft inner tube 112 are each a tubular body in the form of a multilayer coil such as a three-layer coil having alternate winding directions like right-, left- and right-handed winding direction. Examples of the constituent material of the shaft outer tube 111 and the shaft inner tube 112 can include polyolefin such as polyethylene, polypropylene, polyamides, polyesters such as polyethylene terephthalate, fluorine-containing polymers such as ETFE (ethylene-tetrafluoroethylene copolymer), PEEK (polyether ether ketone), polyimides, and combinations thereof, in which a reinforcing member such as a wire-like member may be embedded.

The constituent material of the outermost sheath body 120 is not particularly limited. Examples of the material which can be used preferably include polyolefin such as polyethylene, polypropylene, polyamides, polyesters such as polyethylene terephthalate, fluorine-containing polymers such as ETFE and PEEK, and polyimides. In addition, the outermost sheath body 120 may be composed of a plurality of materials, and a reinforcing member such as a wire-like member may be embedded in the outermost sheath body 120.

A stirring portion 113 is provided at a distal portion of the shaft outer tube 111. The stirring portion 113 has base portions 113A fixed to a peripheral surface of the shaft outer tube 111, at two positions, or on a proximal side and on a distal side, and a plurality of spiral portions 1138 are arranged between these base portions 113A. The spiral portions 1138 can be twisted toward the same direction as viewed along the axial direction, fixing positions at which the spiral portions 1138 are fixed to the base portion 113A differ in the circumferential direction, and axial positions at which the spiral portions 1138 are bent are different; as a whole, the stirring portion 113 is formed in such a shape as to have a uniform swelling in the circumferential direction. As the shaft outer tube 111 is rotated, the stirring portion 113 is also rotated concurrently, whereby a thrombus in a blood vessel can be crushed, or the crushed thrombus can be stirred.

The spiral portions 1138 constituting the stirring portion 113 are composed of flexible thin metal wires. The stirring portion 113 can be kept contained in the inside of the outermost sheath body 120 until the shaft main body 110 is inserted to a target site. After the shaft main body 110 is inserted to a target site, the outermost sheath body 120 is slid toward the proximal side, whereby the stirring portion 113 is exposed to the outside of the outermost sheath body 120, and is expanded into a shape as illustrated in FIG. 8. Therefore, the spiral portions 1138 are desirably formed of a material, which has a shape memory property. As a material of the spiral portions 1138, there can be preferably used, for example, shape memory alloys to which a shape memory effect or hyperelasticity is imparted by a heat treatment, for example, stainless steel. As the shape memory alloys, there can be preferably used Ni—Ti-based, Cu—Al—Ni-based, and Cu—Zn—Al-based alloys, and combinations thereof.

The rotationally driving unit 130 can include a driving motor 131, and a gear part 132 by which the driving motor 131 is interlocked with the shaft outer tube 111 of the shaft main body 110. With the driving motor 131 actuated to rotate, the shaft outer tube 111 can be rotated in the circumferential direction. In this embodiment, the shaft outer tube 111 is driven by the driving motor 131 so as to rotate alternately toward two directions, namely, positive and negative directions, in the circumferential direction. By the alternate rotation toward the two directions, namely, the positive and negative directions, blood flow can be alternately directed in opposite directions.

In the vicinity of a distal portion of the shaft outer tube 111, an opening 160 in a slot form along the axial direction is formed, and the inside and the outside of the shaft outer tube 111 communicate with each other through the opening 160. At a distal portion of the shaft outer tube 111, a cylindrical attaching portion 161 such as to close the hollow inside of the shaft outer tube 111 is provided, whereby a distal portion of the shaft outer tube 111 is closed. A proximal surface of the attaching portion 161 constitutes an attaching face 161A facing a distal surface of the shaft inner tube 112. The attaching face 161A is located on the distal side as compared to a distal-side end portion of the opening 160 of the shaft outer tube 111. The attaching portion 161 can be formed of, for example, stainless steel.

The shaft inner tube 112 has its distal end surface located at the position of, or on the proximal side of the position of, a proximal-side end portion of the opening 160 of the shaft outer tube 111. At a distal-side end portion of the shaft inner tube 112, a cutting unit 162 is provided in the hollow inside of the shaft inner tube 112. The cutting unit 162 is composed of a thin metal sheet, has a width corresponding to the diameter of the shaft inner tube 112, and is formed at the distal end with a sharp blade portion 162A.

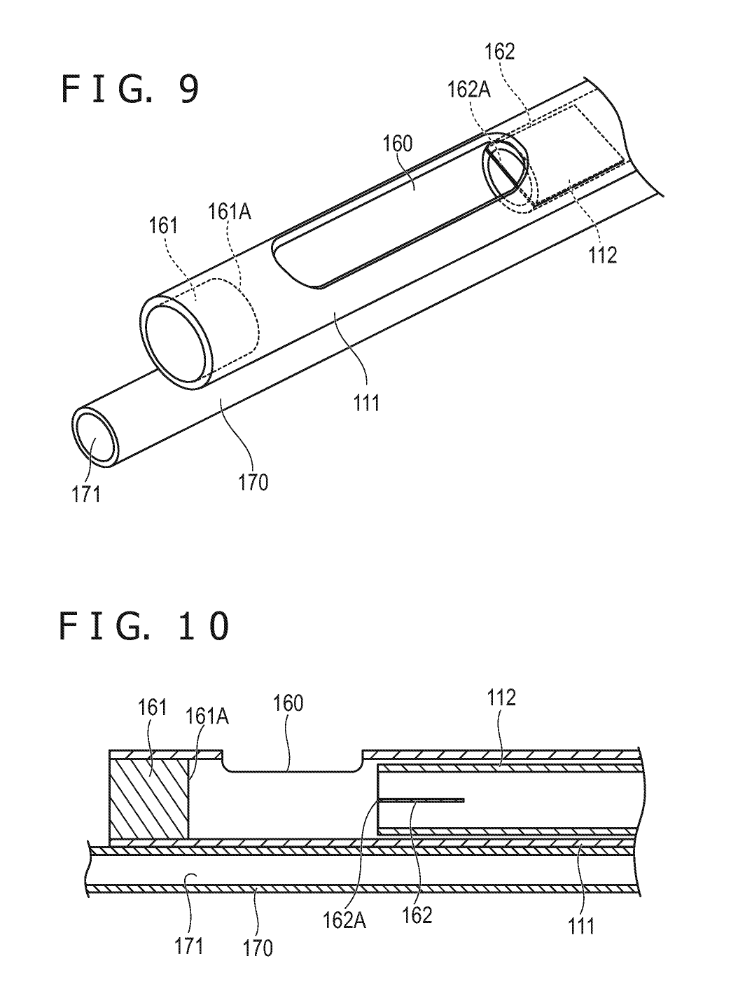

As illustrated in FIG. 9, a distal-side end surface of the blade portion 162A and a distal-side end surface of the shaft inner tube 112 are located in such a manner that no step is formed there. Therefore, when a distal surface of the shaft inner tube 112 makes contact with the attaching face 161A of the attaching portion 161, the blade portion 162A also makes contact with the attaching face 161A. The shaft inner tube 112 can be reciprocated along the axial direction relative to the shaft outer tube 111, at least from the position as illustrated in FIG. 10 to the position of contact with the attaching face 161A of the attaching portion 161. In accordance with an exemplary embodiment, a distal portion of the shaft inner tube 112 may be thinner than the thickness (the thickness obtained by subtracting the inner tube inside diameter from the inner tube outside diameter) of other portions than the distal portion of the shaft inner tube 112, and may have thinness comparable to the thinness of the blade portion 162A of the cutting unit 162.

The shaft outer tube 111 and the shaft inner tube 112 are disposed coaxially, and the shaft outer tube 111 can be reciprocated along the circumferential direction by the rotationally driving unit 130. It is to be noted, however, that the shaft outer tube 111 is not limited to the one that is reciprocated, but may be one that is rotated in one direction. The blade portion 162 is disposed in such a manner as to bisect the cross-sectional shape of the hollow portion of the shaft inner tube 112.

Now, a method of using the medical device 10 and the removal device 100 according to this embodiment will be described below while taking as an example a case of sucking and removing a thrombus present inside a blood vessel.

First, an introducer sheath (not illustrated) is percutaneously inserted into a blood vessel on an upstream side (proximal side) of a thrombus 300 in the blood vessel, and a guide wire 80 is inserted into the blood vessel through the introducer sheath. Next, the guide wire 80 is pushed forward, to reach the distal side of the thrombus 300.

Next, as illustrated in FIG. 2, the medical device 10 having the restriction instrument 20 and the pushing shaft 40 accommodated in the sheath 30 is prepared. The expansion part 22 is disposed at a position near the distal-side end portion of the sheath tubular body 31 of the sheath 30, and its shape is restricted in a contracted state. The shaft part 23 is protruded to the proximal side from the hub opening 35 of the hub 32.

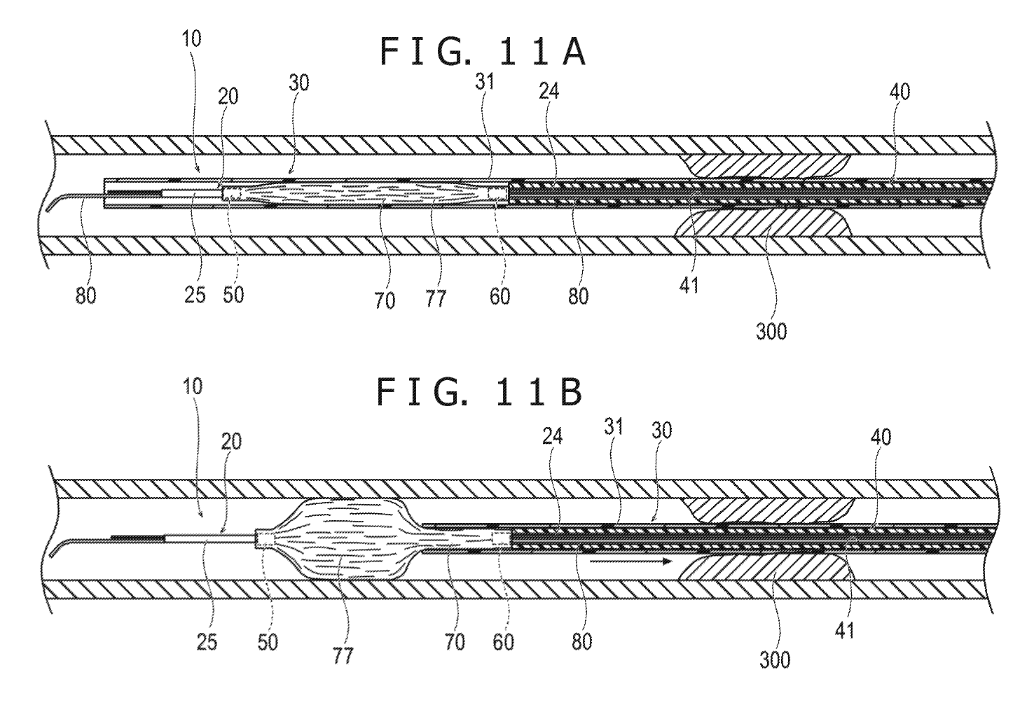

Subsequently, a proximal-side end portion of the guide wire 80 located outside the body is inserted into the guide wire lumen 26 of the medical device 10, and, as illustrated in FIG. 11A, the medical device 10 is moved along the guide wire 80 to reach the distal side of the thrombus 300. Note that in order to cause the guide wire 80 to reach the distal side of the thrombus 300, a support catheter prepared separately may be used.

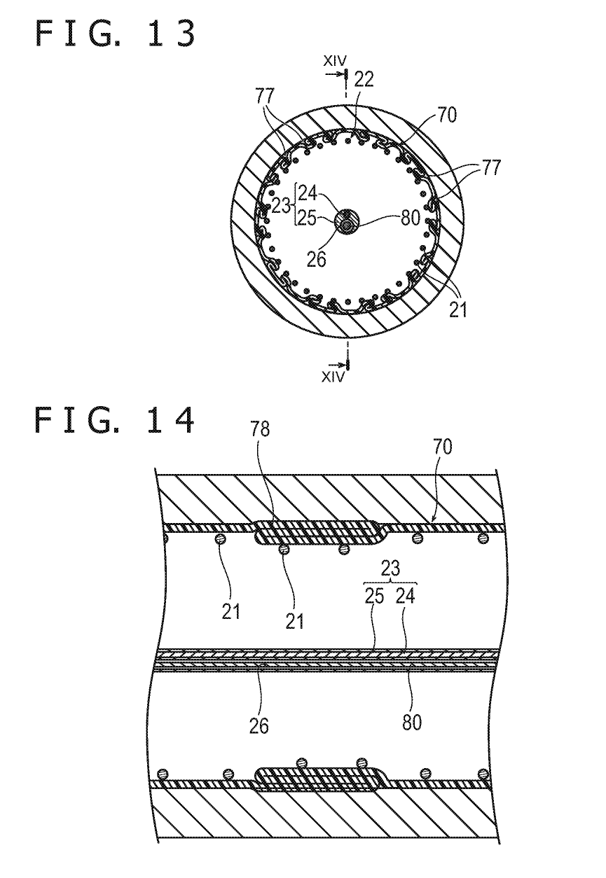

Next, while restricting movement of the pushing shaft 40 by hand, the sheath 30 is moved toward the proximal side. In this instance, the distal-side end portion of the pushing shaft 40 makes contact with the proximal-side connection part 60 or a proximal-side end portion of the guide wire tubular body 25, and movement of the expansion part 22 and the cover part 70 is restricted, so that the positions of proximal portions of the expansion part 22 and the cover part 70 in the blood vessel can be arbitrarily adjusted. Then, the sheath 30 is moved toward the proximal side relative to the pushing shaft 40, whereby the expansion part 22 and the cover part 70 are gradually discharged from the sheath tubular body 31. By this, as illustrated in FIG. 11B, while the distal-side connection part 50 is moved closer to the proximal-side connection part 60, the expansion part 22 is expanded to an optimum size by its own restoring force, and a distal portion of the cover part 70 is pressed against the inner wall surface of the blood vessel and thereby positioned. Since the expansion part 22 is formed in a mesh shape, it can firmly fix the cover part 70 while causing the cover part 70 to bite into the inner wall surface of the blood vessel. The cover part 70 is forced open while the folded-back parts 77 are stretched by the expansion part 22, according to the inside diameter and shape of the blood vessel, and the cover part 70 is pressed against, and caused to contact, the inner wall surface of the blood vessel by the expansion part 22. Note that in the state in which the cover part 70 is in contact with the inner wall surface of the blood vessel, even if the folded-back parts 70 in a folded-back state are remaining, as illustrated in FIG. 13, the cover part 70 is pressed against the inner wall surface of the blood vessel by the expansion part 22, so that no gap is generated between the cover part 70 and the blood vessel. Since the plurality of folded-back parts 77 provided in the cover part 70 are formed intermittently and shortly in the axial direction, minute gaps generated between the folded-back parts 77 and the inner wall surface of the blood vessel are also formed intermittently in the axial direction and are not formed continuously in the axial direction of the cover part 70. Therefore, a flow of blood can be effectively restrained by the cover part 70.

In addition, the expansion part 22 is accommodated in the cover part 70 while being forcibly restrained by the cover part 70 from expanding in diameter. Therefore, not only in the case where the outside diameter in an expanded state is small but also in the case where the outside diameter in an expanded state is large, the expansion part 22 can exhibit a sufficient expanding force against the inner wall surface of the blood vessel and can achieve firm fixation to the inner wall surface. Thus, the range of the inside diameter of the blood vessel to which the medical device 10 can be applied is wide.

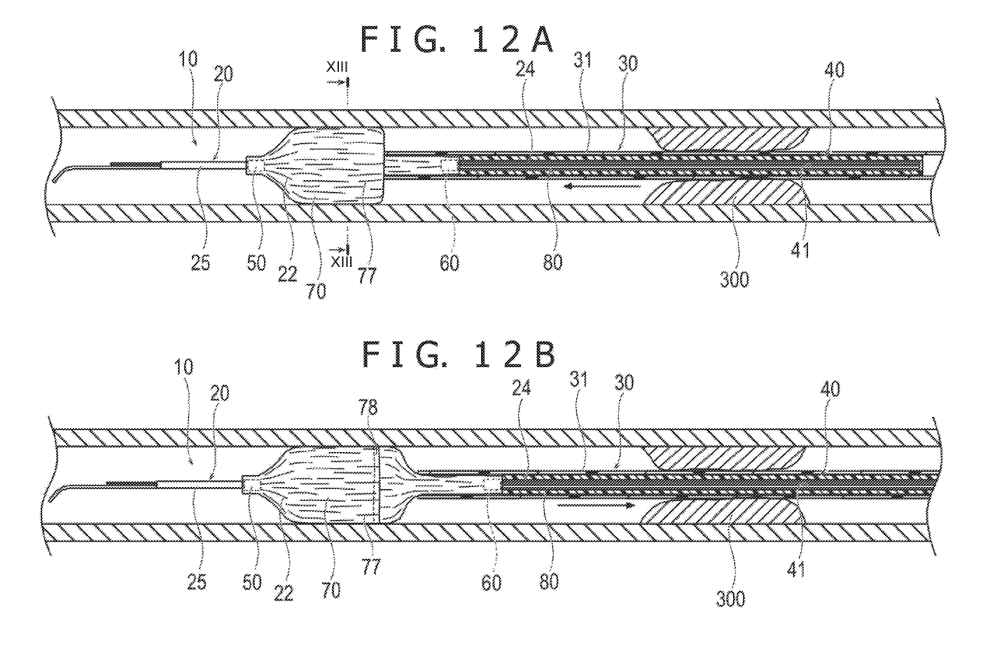

Subsequently, the pushing shaft 40 is pushed in, whereby a portion on the proximal side of that portion of the cover part 70 which is discharged from the sheath 30 is pushed in to the inside of the cover part 70 on the distal side, as illustrated in FIG. 12A. In this instance, the sheath 30 may be pushed in together with the pushing shaft 40, without being moved relative to the pushing shaft 40, or may be moved toward the proximal side relative to the pushing shaft 40. In the case where the sheath 30 is pushed in together with the pushing shaft 40, those portions of the cover part 70 and the expansion part 22 which are accommodated in the sheath tubular body 31 are not pushed out to the exterior, and those portions of the cover part 70 and the expansion part 22 which have already been located outside of the sheath tubular body 31 are pushed in to the distal side. In the case where the pushing shaft 40 is pushed in while the sheath 30 is being moved toward the proximal side relative to the pushing shaft 40, the cover part 70 and the expansion part 22 are pushed in to the distal side, while those portions of the cover part 70 and the expansion part 22 which are accommodated in the sheath tubular body 31 are newly pushed out from the sheath tubular body 31 to the distal side.

Next, the pushing-in of the pushing shaft 40 is stopped, and, while restricting movement of the pushing shaft 40 by hand, the sheath 30 is moved toward the proximal side relative to the pushing shaft 40. By this, the expansion part 22 and the cover part 70 can be gradually released from the sheath tubular body 31, and an overlapping part 78 projecting to the proximal side at the outer peripheral surface is formed, as illustrated in FIGS. 12B and 14. In accordance with an exemplary embodiment, the axial length of the overlapping part 78 can be arbitrarily set by the pushing-in length of the pushing shaft 40.

After the expansion part 22 and the cover part 70 are pushed out from the sheath tubular body 31 to a certain extent, the pushing shaft 40 is again pushed in, then the pushing-in is stopped, and the expansion part 22 and the cover part 70 are released from the sheath tubular body 31, whereby an overlapping part 78 can again be formed at an arbitrary position. Only one overlapping part 78 may be formed, or a plurality of overlapping parts 78 may be formed. In this embodiment, three overlapping parts 78 are formed, and the overlapping part 78 on the more proximal side is longer in the axial direction. Therefore, a blood flow restraining effect produced by the overlapping part 78 is greater on the upstream side of the blood vessel, so that blood flow can be restrained effectively. The overlapping parts 78 are preferably disposed biasedly to the proximal side, but they may be disposed biasedly to the distal side, or they may be disposed non-biasedly. In this embodiment, the overlapping parts 78 are disposed biasedly to the proximal side, the blood flow restraining effect produced by the overlapping part 78 is greater on the upstream side of the blood vessel, so that blood flow can be restrained effectively.

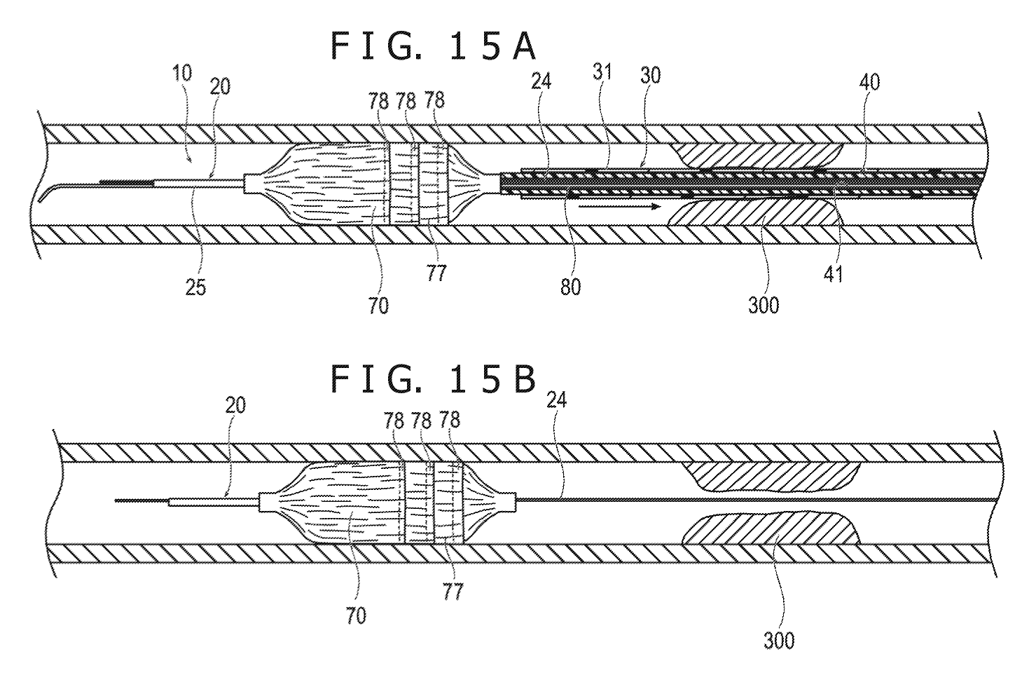

After a desired number of overlapping parts 78 are formed, the sheath 30 is moved toward the proximal side relative to the pushing shaft 40 while restricting movement of the pushing shaft 40 by hand, whereby the expansion part 22 and the cover part 70 are released completely from the sheath tubular body 31. Since the maximum diameter to which the expansion part 22 being used can be expanded is greater than the diameter of the blood vessel into which the medical device 10 is inserted, the expansion part 22 is not fully expanded inside the blood vessel. Therefore, the expansion part 22 can generate an expanding force, and can effectively bring the cover part 70 into secure contact with the blood vessel wall. As a result, as illustrated in FIG. 15A, the cover part 70 is pressed against the inner wall surface of the blood vessel by the expansion part 22 and thereby fixed inside the blood vessel. Thereafter, as illustrated in FIG. 15B, the sheath 30 and the pushing shaft 40 are discharged to the outside of the body, leaving the restriction instrument 20 in the blood vessel.

When the expansion part 22 and the cover part 70 are put in secure contact with the inner wall surface of the blood vessel, blood flow in the blood vessel is intercepted or reduced, and blood stays still. In addition, the cover part 70 is formed with a plurality of overlapping parts 78, and the portions where the overlapping parts 78 are formed are increased in wall thickness and project radially. Therefore, the restraining effect on the blood flow about to occur between the blood vessel and the cover part 70 can be enhanced by the overlapping parts 78. In addition, since the overlapping parts 78 are so formed as to project toward the proximal side at the outer circumferential face of the cover part 70, the blood flow restraining effect produced by the overlapping parts 78 can be further enhanced. Furthermore, since the axial length of the overlapping part 78 on the more proximal side of the cover part 70 is greater than that on the more distal side and the overlapping parts 78 are disposed biasedly to the proximal side, the blood flow restraining effect can be enhanced on the upstream side of the blood vessel. Accordingly, while effectively restraining the blood flow, the cover part 70 can be restrained from being elongated more than necessary due to the provision of too large a number of the overlapping parts 78.

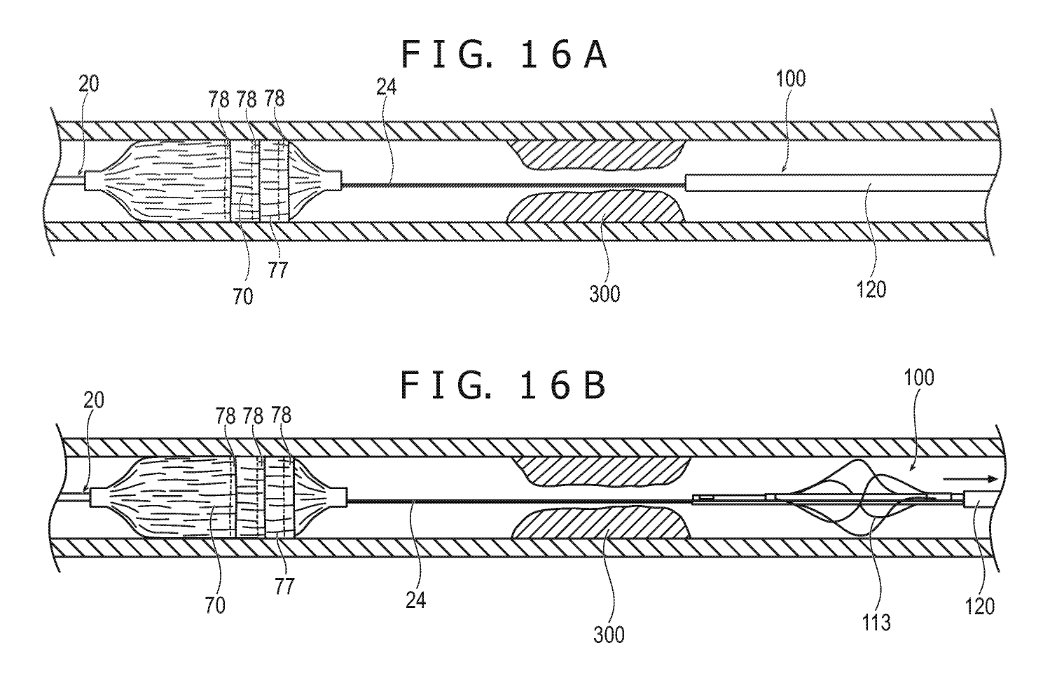

Next, the removal device 100 in a state in which a distal portion of the shaft main body 110 including the stirring portion 113 is accommodated in the outermost sheath body 120 is prepared, and a proximal-side end portion of the wire part 24 is inserted into the second guide wire lumen 171 of the removal device 100. Thereafter, as illustrated in FIG. 16A, the removal device 100 is inserted to the proximal side of the thrombus 300, with the wire part 24 as a guide. Then, the outermost sheath body 120 is moved toward the proximal side, whereon the stirring portion 113 is spread inside the blood vessel, as depicted in FIG. 16B.

Subsequently, utilizing the outermost sheath body 120, the shaft inner tube 112 or the second guide wire lumen 171 (see FIG. 9), a thrombolytic agent is injected into the vicinity of the thrombus 300 in the blood vessel. In this instance, since blood flow in a region where the thrombus is formed is restricted (intercepted or reduced), the thrombolytic agent is kept in a high concentration, and exhibits a high thrombolytic effect. Note that the thrombolytic agent may not necessarily be used.

Next, in a state in which the stirring portion 113 has been moved forward into the vicinity of the thrombus 300, the shaft outer tube 111 is rotated by the rotationally driving unit 130, whereon the stirring portion 113 is also rotated attendantly, and the thrombus 300 in a firmly adhered state inside the blood vessel is crushed.

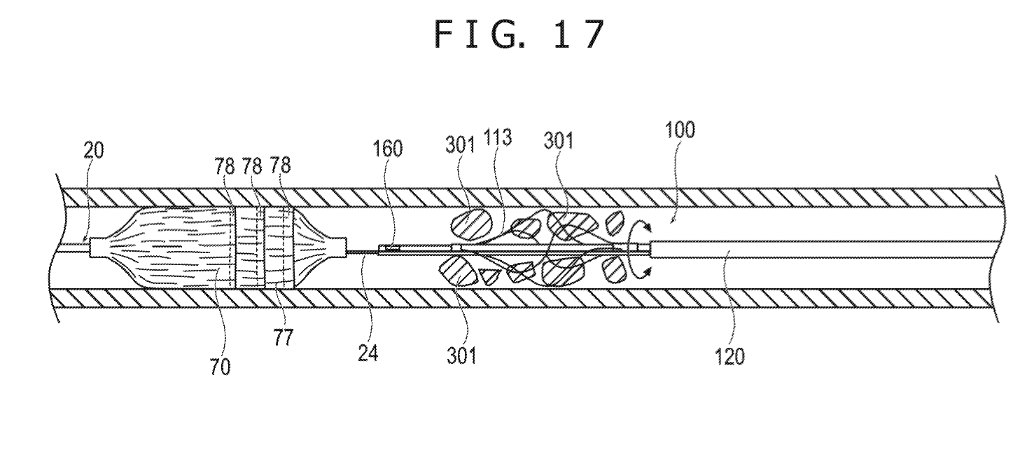

When the rotation of the stirring portion 113 is continued, the thrombus 300 firmly adhered inside the blood vessel is entirely crushed, and a crushed thrombus 301 is put into a floating state without precipitation in the blood vessel in which it is stagnating, as illustrated in FIG. 17, since the flow of blood is restricted by the medical device 10.

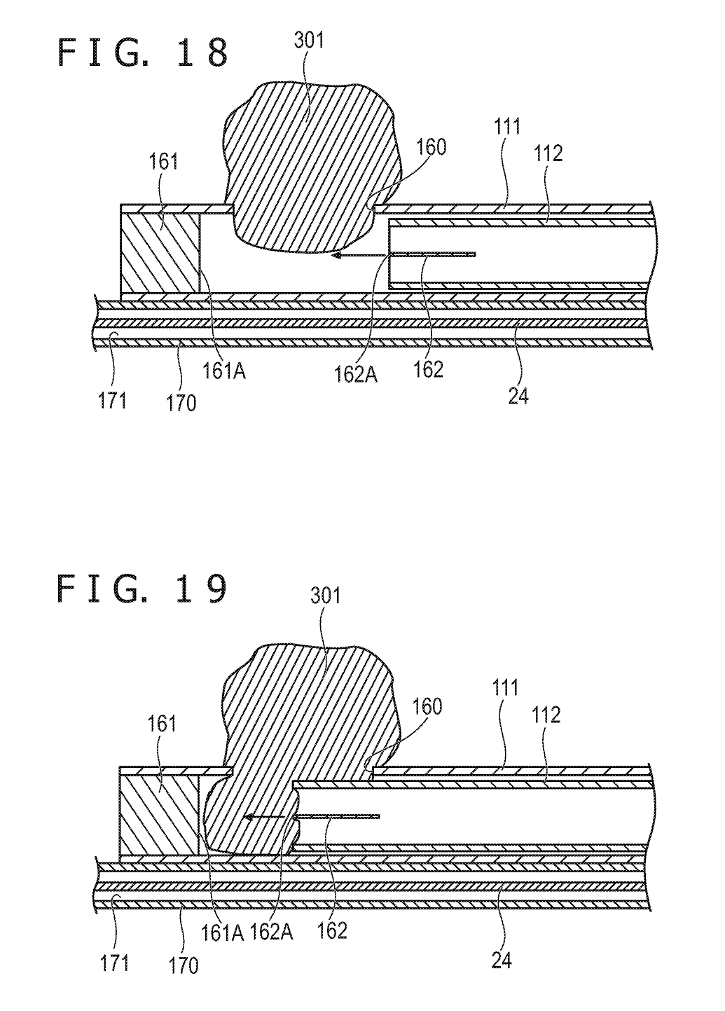

Next, a pusher of the syringe 150 (see FIG. 8) is pulled, to bring the hollow inside of the shaft inner tube 112 into a negative pressure state. Here, a distal-side end portion of the shaft inner tube 112 communicates with the hollow inside of the shaft outer tube 111, and, further, the shaft outer tube 111 communicates with the exterior of the shaft main body 110 through the opening 160. Therefore, the opening 160 generates a suction force exerted to the exterior of the shaft main body 110, whereby the crushed thrombus 301 floating inside the blood vessel is drawn. As illustrated in FIG. 18, the thrombus 301 drawn to the opening 160 partly enters into the hollow inside of the shaft outer tube 111.

After the pusher of the syringe 150 is pulled, the shaft inner tube 112 is moved in the axial direction relative to the shaft outer tube 111. When the shaft inner tube 112 is moved toward the distal side of the shaft outer tube 111, namely, toward the side of approaching the attaching portion 161, from a state in which the shaft inner tube 112 is located on the proximal side of the opening 160, that part of the thrombus 301 which has entered into the hollow inside of the shaft outer tube 111 through the opening 160 is gradually cut off while being compressed by a distal surface of the shaft inner tube 112, as illustrated in FIG. 19.

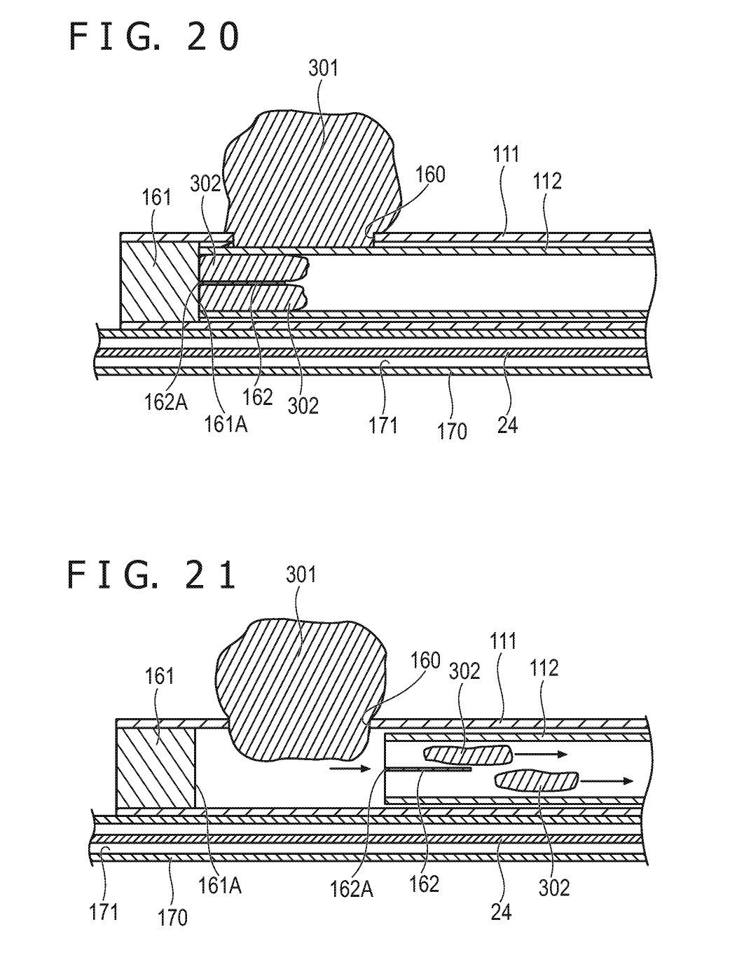

When the shaft inner tube 112 is moved until the distal surface of the shaft inner tube 112 makes contact with the attaching face 161A of the attaching portion 161, a thrombus piece 302 cut off is accommodated in the hollow inside of the shaft inner tube 112, as illustrated in FIG. 20. In this instance, the thrombus piece 302 is bisected by the blade portion 162A of the cutting unit 162 provided at a distal portion of the shaft inner tube 112. With the shaft inner tube 112 making contact with the attaching face 161A of the attaching portion 161, the blade portion 162A also makes contact with the attaching face 161A, and the thrombus piece 302 cut off in the hollow inside of the shaft outer tube 111 is cut by the blade portion 162A while being pressed against the attaching portion 161. Therefore, the thrombus piece 302 can be securely cut off into a size smaller than the inside diameter of the shaft inner tube 112. As a result, the cut-off thrombus piece 302 can be restrained from clogging in the hollow inside of the shaft inner tube 112.

Since the hollow inside of the shaft inner tube 112 is continuously kept in the negative pressure state by the syringe 150, the cut-off thrombus pieces 302 are moved within the hollow inside of the shaft inner tube 112 toward the proximal side, as illustrated in FIG. 21. In addition, with the shaft inner tube 112 separated from the attaching portion 161 and moved toward the proximal side, the opening 160 is opened again, the thrombus 301 is sucked and enters into the hollow inside of the shaft outer tube 111. Therefore, by repeating reciprocation of the shaft inner tube 112 in the axial direction, the thrombus 301 can be continuously sucked while being cut into small pieces.

While the crushed thrombus 301 is sucked by the shaft main body 110, a rotating operation of the shaft outer tube 111 is desirably continued. With the shaft outer tube 111 rotating, a vortex is generated in the blood inside the blood vessel, and the thrombus 301 is liable to gather in the vicinity of a rotational center, namely, in the vicinity of a radial center of the blood vessel; accordingly, the thrombus 301 can be relatively easily sucked via the opening 160. In addition, the vortex generated in the vicinity of the opening 160 influences the flow in the hollow inside of the shaft inner tube 112, and a swirling flow of vortex is generated also in the inside of the shaft inner tube 112. As a result, flow resistance in the axial direction can be reduced inside the shaft inner tube 112, and the cut thrombus pieces 302 can be smoothly sucked.

In this embodiment, during suction of the thrombus 301, the shaft outer tube 111 is rotated, and the shaft inner tube 112 is reciprocated in the axial direction relative to the shaft outer tube 111; however, other operation or operations may be added. For example, an operation in which the shaft inner tube 112 performs rotation relatively different from the rotation of the shaft outer tube 111 (rotating directions are reverse to each other, or the rotation directions are the same but rotating speeds are different) may be added, whereby the thrombus 301 sucked into the opening 160 can be securely cut off and be led into the hollow inside of the shaft outer tube 111. In addition, reciprocation of the shaft outer tube 111 may be added, whereby the thrombus 300 in a wider range can be crushed and/or stirred.

In this embodiment, since a flow of blood is restricted by the medical device 10, the crushed thrombus 301 floats in the stagnating blood. Therefore, the thrombus 301 can be efficiently sucked via the opening 160 and be removed from inside the blood vessel, without permitting the thrombus 301 to flow to other part. In addition, while a strong suction force is required where blood is flowing, blood flow is restrained in this embodiment, so that a suction force acts relatively easily and the thrombus 301 can be sucked more effectively in this embodiment.

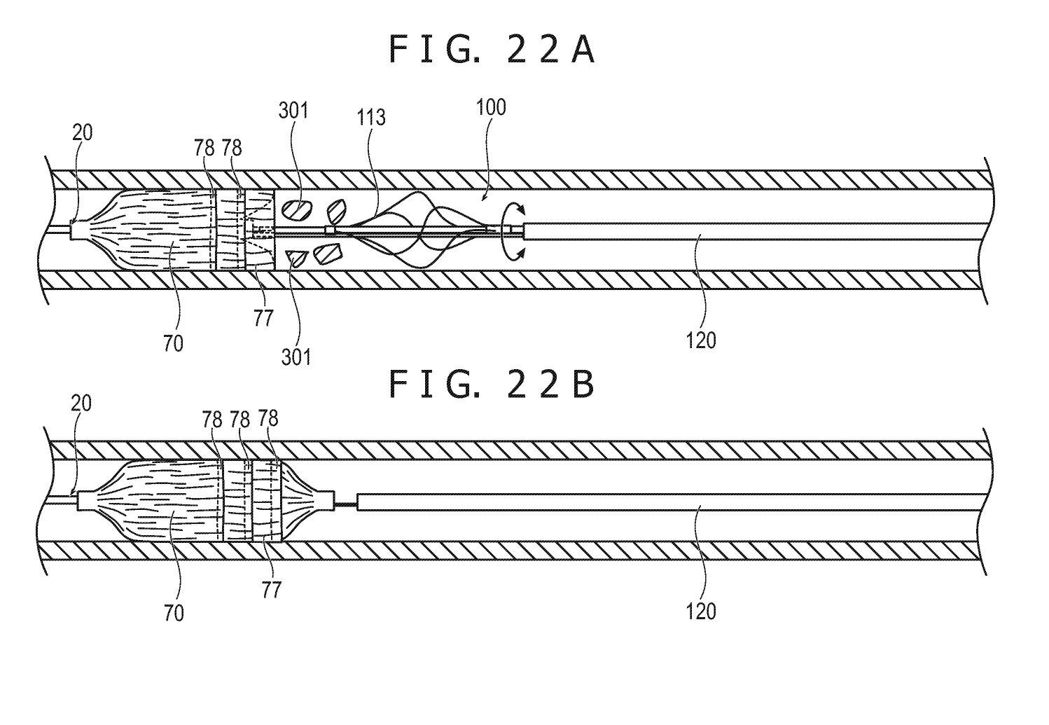

In addition, a configuration may be adopted in which as illustrated in FIG. 22A, the removal device 100 is pressed against the cover part 70 at the time of suction of the thrombus 301; for example, the thrombus 301 adhering to the cover part 70 can be sucked via the opening 160, while denting a proximal portion of the cover part 70.

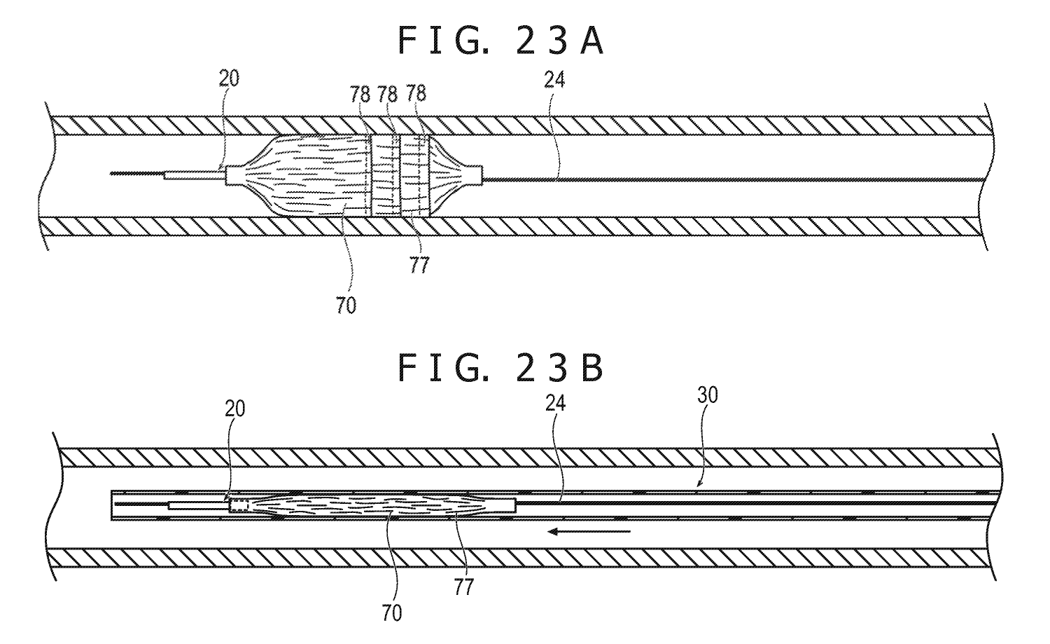

After the suction of the thrombus 301 is completed, the reciprocation and rotation of the shaft outer tube 111 and the shaft inner tube 112 are stopped, and the outermost sheath body 120 is moved in the axial direction to accommodate the stirring portion 113 therein, as illustrated in FIG. 22B. Thereafter, the removal device 100 is pulled out of the blood vessel, leaving the restriction instrument 20 in the blood vessel, as illustrated in FIG. 23A.

Next, a proximal-side end portion of the wire part 24 is inserted into the sheath 30, the sheath 30 is inserted into the blood vessel along the wire part 24, and the sheath 30 is made to reach the vicinity of the expansion part 22 and the cover part 70. Subsequently, while grasping the proximal-side end portion of the wire part 24 and restraining axial movement thereof, the sheath 30 is pushed in, as illustrated in FIG. 23B, whereby the expansion part 22 and the cover part 70 are accommodated into the inside of the sheath 30 while being contracted in diameter. When the cover part 70 is contracted in diameter, the blood inside the cover part 70 is discharged via the hole portions 76 to the exterior. When the cover part 70 is accommodated into the inside of the sheath 30, the thrombus 301 adhering to the cover part 70 can also be accommodated into the sheath 30. In addition, a configuration can be adopted in which the cover part 70 making contact with the inner wall surface of the blood vessel is moved in the proximal direction, to rub off the thrombus 300 adhering to the blood vessel by the cover part 70, after which the thrombus 300 is accommodated into the sheath 30 together with the cover part 70. Thereafter, the restriction instrument 20 is pulled out of the blood vessel together with the sheath 30, whereby the treatment is completed.

As has been described above, the medical device 10 according to this embodiment is a medical device 10 for restricting a flow in a body lumen by being inserted into the body lumen. The medical device can include: an elongated shaft part 23; an expansion part 22 which is a tubular body having a plurality of gaps 21A and elastically deformable, an outside diameter of the tubular body being greater at a central portion than both side end portions of the tubular body in a natural state where no external force is acting, with the shaft part 23 being connected to at least one of the both side end portions of the tubular body; a cover part 70 which is flexibly deformable independently from the expansion part 22 while surrounding an outer circumference of the expansion part 22, which is tubular in shape, both end portions of the tubular shape being connected to both end portions of the expansion part 22, and which is capable of forming an overlapping part 78 overlapping by being folded back in an axial direction such that inner surface portions of the tubular shape contact each other when the expansion part 22 is expanded; and a sheath 30 in which the expansion part 22 and the cover part 70 both contracted in diameter can be accommodated. In the medical device 10 configured as above, with the expansion part 22 and the cover part 70 released from the sheath 30, the expansion part 22 expands in conformity with the shape of a body lumen by its own elastic force, the overlapping part 78 projecting in the radial direction by overlapping is formed along the whole circumference of the cover part 70, and, concurrently, the cover part 70 is pressed against the body lumen by the expansion part 22. Therefore, the range of inside diameter of the body lumen to which the medical device 10 is applicable is widened by the expansion part 22 which expands by its own elastic force; in addition, a flow in the body lumen can be effectively restricted by the cover part 70 formed with the overlapping part 78, and a thrombus 300 (substance) can be effectively sucked from inside the body lumen.

In addition, in the medical device 10, the expansion part 22 is formed by knitting a plurality of elastically deformable wire-like members 21 into a tubular form. By this it can be ensured that, while permitting the expansion part 22 to be expanded to have a larger outside diameter by its own elastic force, the expansion part 22 becomes shorter in the axial direction when expanded since the expansion part 22 is knitted, and the axial length of a portion which becomes the overlapping part 78 can be relatively easily secured in the cover part 70.

In addition, in the medical device 10, the cover part 70 is formed with at least one hole portion 76 at least on only the distal side of cover part 70. By this, flow-in of blood (fluid) into the cover part 70 and discharge of the blood from the cover part 70 to the exterior through the hole portion 76 can be performed, and a variation in volume of the cover part 70 can be easily generated.

In addition, in the medical device 10, the cover part 70 is capable of forming the folded-back part 77 overlapping by being folded back in a circumferential direction such that inner surface portions contact each other. By this it can be ensured that when the cover part 70 contacts a body lumen, an increased contact area is obtained, an enhanced contact force is obtained, and a flow of blood can be restrained effectively. In addition, with the cover part 70 formed with both the overlapping parts 78 and the folded-back parts 77, an outer surface of the cover part 70 is pressed against the body lumen by the expansion part 22 while being partitioned into sections in a roughly grid-like pattern, so that movement of blood between the partitioned sections is restrained, and a flow of blood can be restrained effectively. Note that while the folded-back parts 77 and the overlapping parts 78 are formed by folding back of a blank material of the cover part 70, the folded-back parts 77 and the overlapping parts 78 can be separated from the expansion part 22, so that the expansion part 22 making contact with the folded-back parts 77 and the overlapping parts 78 is not folded back.

In addition, in the medical device 10, the position at which the cover part 70 contacts the expansion part 22 when contracted and the position at which the cover part 70 contacts the expansion part 22 when expanded are different from each other. This helps ensure that the cover part 70 can form the folded-back parts 77 and the overlapping parts 78 by deforming independently from the expansion part 22, and intersection angles of the wire-like members 21 constituting the expansion part 22 can vary without being hampered by the cover part 70, so that the expansion part 22 can be deformed flexibly.

In addition, in the medical device 10, at least part of the folded-back part 77 extending in the axial direction when the cover part 70 is contracted is deformable into the overlapping part 78 when the cover part 70 is expanded. By this, it can help ensure that the folded-back parts 77 formed when the cover part 70 is contracted are reduced in number or amount by expansion of the cover part 70, and, concurrently, at least part of the folded-back parts 77 becomes the overlapping parts 78. Accordingly, the cover part 70 can be deformed according to the situation, and the cover part 70 can be utilized efficiently.

In addition, in the medical device 10, the expansion part 22 is formed by knitting of a plurality of wire-like members 21B and 21C which differ in outside diameter. This helps ensure that when the expansion part 22 is contracted and accommodated in the sheath 30, the thinner wire-like member 21C is not liable to contact the inner wall surface of the sheath 30 through the cover part 70. Therefore, the positions of the intersections of the netting are not liable to become irregular, the shape of the expansion part 22 is stabilized, and a flow of blood can be restrained effectively.

In addition, the cover part 70 may have a preliminary shape portion 79 which is preliminarily shaped for promoting the formation of the overlapping part 78. Accordingly, the overlapping part 78 can be easily formed inside the body lumen.

In addition, the medical device 10 has the pushing shaft 40 which is a tubular body accommodated in the sheath 30 and penetrated by the shaft part 23, which is formed to have an inside diameter such that the expansion part 22 and the cover part 70 in the sheath 30 cannot pass therethrough, and which pushes out the expansion part 22 and the cover part 70 from the sheath 30. This helps ensure that by utilizing the pushing shaft 40, the expansion part 22 and the cover part 70 can be easily pushed out from inside the sheath 30.

In addition, the present disclosure provides a treatment method for sucking and removing a substance generated at a lesion in a body lumen by utilizing the aforementioned medical device. The method can include: a step of gradually pushing out the expansion part 22 and the cover part 70 from the sheath 30 to a downstream side with respect to the lesion in the body lumen, and expanding the expansion part 22 by its own elastic force, to thereby cause the cover part 70 to contact the body lumen from a distal side; a step of pushing in the expansion part 22 and the cover part 70 in a distal direction, and thereafter further pushing out the expansion part 22 and the cover part 70 from the sheath 30, to thereby fold back the cover part 70 in an axial direction and form an overlapping part 78; a step of crushing or dissolving the substance generated at the lesion in the body lumen; a step of inserting into the body lumen a device provided with a suction port capable of suction, to suck the crushed or dissolved substance; a step of contracting the expansion part 22 and the cover part 70; and a step of drawing out the medical device 10 from inside the body lumen. In the treatment method configured in this way, the overlapping part 78 is formed while pushing the expansion part 22 and the cover part 70 in the distal direction, and, therefore, the overlapping part 78 can be freely formed with an arbitrary axial length. Accordingly, the overlapping part 78 increased in material thickness and projecting in the radial direction due to the overlapping can be formed with a desirable axial length and along the whole circumference of the cover part 70. Thus, flow in the body lumen can be effectively restrained by the cover part 70 formed with the overlapping part 78, and the substance can be effectively sucked from inside the body lumen.

In addition, in the step of forming the overlapping part 78, the sheath 30 is moved in the proximal direction relative to the expansion part 22 and the cover part 70, the distal side of the cover part 70 is caused to contact the inner wall of the body lumen, and thereafter the expansion part 22 and the cover part 70 are pushed out in the distal direction by the pushing shaft 40, the cover part 70 can be folded back in the axial direction. As a result, the cover part 70 can be easily formed with the folded-back part 78 by an operation on the hand side.

In addition, in the step of forming the overlapping part 78, the cover part 70 can be folded back in such a manner that the overlapping part 78 projects toward the proximal side at the outer circumferential face of the cover part 70. This helps ensure that a flow in the body lumen is effectively restrained by the overlapping part 78, whereby the substance can be effectively sucked from inside the body lumen. Note that the overlapping part 78 may be formed such as to project toward the distal side at the outer circumferential face of the cover part 70. According to such a configuration, the cover part 70 can be easily retracted into the sheath 30 after expanded in the blood vessel.

Further, in the step of forming the overlapping part 78, a plurality of overlapping parts 78 can be formed in the axial direction, and the axial length of the overlapping part 78 located on the more proximal side can be set greater than the axial length of the overlapping part 78 located on the more distal side. As a result, the blood flow restraining effect produced by the overlapping part 78 is greater on the upstream side of the blood vessel, and the blood flow can be restrained effectively.

Note that the present disclosure is not limited only to the above-described embodiment, and various modifications are possible by a person skilled in the art within the technical thought of the present disclosure. For instance, while the medical device 10 is structured to obtain access to a target site from the upstream side of the patient in this embodiment, a structure for obtaining access to the target site from the downstream side of the patient may be adopted.

In addition, while the thrombus 300 is crushed by the removal device 100 having the stirring portion 113 in this embodiment, the medical device 10 may be used not for crushing the thrombus 300 but for effectively dissolving the thrombus 300 by a thrombolytic agent. With the blood flow restricted by use of the medical device 10, the thrombolytic agent can be kept staying around the thrombus 300, whereby the thrombus 300 can be dissolved effectively.

In addition, the body lumen into which the medical device 10 is to be inserted is not restricted to a blood vessel, and may, for example, be a vessel, ureter, biliary duct, fallopian tube, hepatic duct, or the like. In addition, the removal device is not limited to the aforementioned structure.

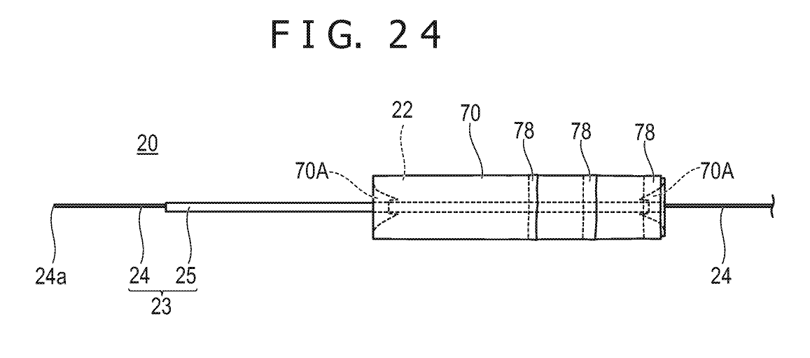

While the expansion part 22 and the cover part 70 are formed to be tapered at axially end portions in this embodiment, hollowed portions 70A hollowed toward the inside of the cover part 70 may be formed at both end portions, as illustrated in FIG. 24. With such a configuration, the axial lengths of the expansion part 22 and the cover part 70 can be made as small as possible, and these parts can be disposed compactly into the target position. As a result, the range of cutting by the stirring portion 113 can be widely secured. In addition, on the proximal side, the thrombus pieces 302 can be favorably retained by the hollow portion 70A. Note that the hollow portion 70A may be formed only on one end side of the cover part 70.

In addition, at least part of the distal-side connection part 50, the proximal-side connection part 60 and the wire-like members 21 may be formed with a radioscopic contrast material contained in the material thereof. For example, part of the plurality of wire-like members 21 may be formed with a radioscopic contrast material contained in the material thereof. This helps enable accurate grasping of position under radioscopy, leading to an easier operation. Preferable examples of the radioscopic contrast material include gold, platinum, platinum-iridium alloys, silver, stainless steel, molybdenum, tungsten, tantalum, palladium, and alloys thereof.

Having described the preferred embodiment of the present disclosure with reference to the accompanying drawings, it is to be understood that the disclosure is not limited to the precise embodiment and that various changes and modifications could be effected therein by one skilled in the art without departing from the spirit or scope of the disclosure as defined by the appended claims.

The detailed description above describes a medical device and treatment method. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device for restricting a flow in a body lumen by being inserted into the body lumen, the medical device comprising:
an elongated shaft part;
an expansion part which is a tubular body having a plurality of gaps and elastically deformable, an outside diameter of the tubular body being greater at a central portion than at both side end portions of the tubular body in a natural state where no external force is acting, with the shaft part being connected to at least one of the both side end portions of the tubular body;
a cover part which is flexibly deformable independently from the expansion part while surrounding an outer circumference of the expansion part, which is tubular in shape, both end portions of the tubular shape being connected to both end portions of the expansion part, and which is capable of forming an overlapping part overlapping by being folded back in an axial direction such that inner surface portions of the tubular shape contact each other when the expansion part is expanded; and
a sheath in which the expansion part and the cover part both contracted in diameter are accommodated.

2. The medical device according to claim 1, wherein the expansion part is formed by knitting a plurality of elastically deformable wire-like members into a tubular form.

3. The medical device according to claim 1, wherein the cover part is formed with at least one hole portion at least on only a distal side of the cover part.

4. The medical device according to claim 1, wherein the cover part is capable of forming a folded-back part overlapping by being folded back in a circumferential direction such that inner surface portions contact each other.

5. The medical device according to claim 1, wherein the cover part is configured such that a position at which the cover part contacts the expansion part when contracted and a position at which the cover part contacts the expansion part when expanded are different from each other.

6. The medical device according to claim 1, wherein at least part of the folded-back part extending in the axial direction when the cover part is contracted is deformable into the overlapping part when the cover part is expanded.

7. The medical device according to claim 1, further comprising:
a pushing shaft which is a tubular body accommodated in the sheath and penetrated by the shaft part, which is formed to have an inside diameter such that the expansion part and the cover part in the sheath cannot pass therethrough, and which pushes out the expansion part and the cover part from the sheath.

8. The medical device according to claim 1, wherein the tubular body of the expansion part includes a distal-side connection part and a proximal-side connection part which are connected to the elongated shaft part, and wherein one of the distal-side connection part and a proximal-side connection part is fixed to the elongated shaft part and another one of the distal-side connection part and the proximal-side connection part is slidable on the elongated shaft part.

9. A medical device for restricting a flow in a body lumen by being inserted into the body lumen, the medical device comprising:
an elongated shaft;
an expandable tubular body having a plurality of gaps and elastically deformable, the expandable tubular body having a central portion, a tapered proximal-side portion, and a taper distal-side portion, and the elongated shaft being connected to at least one end portion of the expandable tubular body; and
a tubular cover which is flexibly deformable independently from the expandable tubular body while surrounding an outer circumference of the expandable tubular body, both end portions of the tubular cover being connected to both end portions of the expandable tubular body, and which is capable of forming an overlapping part overlapping by being folded back in an axial direction such that inner surface portions of the tubular cover contact each other when the expandable tubular body is expanded.

10. The medical device according to claim 9, further comprising:
a sheath configured to accommodate the expandable tubular body and the tubular in a contracted diameter state; and
a pushing shaft which is a tubular body accommodated in the sheath and penetrated by the elongated shaft, which is formed to have an inside diameter such that the expandable tubular body and the tubular cover in the sheath cannot pass therethrough, and which pushes out the expandable tubular body and the tubular cover from the sheath.

11. The medical device according to claim 9, wherein the expandable tubular body is formed by knitting a plurality of elastically deformable wires into a tubular form.

12. The medical device according to claim 9, wherein the tubular cover includes at least one hole portion on a tapered distal side of the tubular cover.

13. A treatment method for sucking and removing a substance generated at a lesion in a body lumen by use of a medical device, the medical device including an elongated shaft part, an expansion part which is a tubular body having a plurality of gaps and elastically deformable, an outside diameter of the tubular body being greater at a central portion than at both side end portions of the tubular body in a natural state where no external force is acting, with the shaft part being connected to at least one of the both side end portions of the tubular body, a cover part which is flexibly deformable independently from the expansion part while surrounding an outer circumference of the expansion part, which is tubular in shape, both end portions of the tubular shape being connected to both end portions of the expansion part, and which is capable of forming an overlapping part overlapping by being folded back in an axial direction such that inner surface portions of the tubular shape contact each other when the expansion part is expanded, and a sheath in which the expansion part and the cover part both contracted in diameter are accommodated, the treatment method comprising:
gradually pushing out the expansion part and the cover part from the sheath to a downstream side with respect to the lesion in the body lumen, and expanding the expansion part by its own expanding force, to cause the cover part to contact the body lumen from a distal side;
pushing in the expansion part and the cover part in the distal direction and thereafter further pushing out the expansion part and the cover part from the sheath, to thereby fold back the cover part in the axial direction and form an overlapping part;
crushing or dissolving the substance generated at the lesion in the body lumen;
inserting into the body lumen a device provided with a suction port capable of suction to suck the crushed or dissolved substance;
contracting the expansion part and the cover part; and
drawing out the medical device from inside the body lumen.

14. The treatment method according to claim 13, wherein in the forming the overlapping part the sheath is moved in a proximal direction relative to the expansion part and the cover part, the distal side of the cover part is made to contact an inner wall of the body lumen, and thereafter the expansion part and the cover part are pushed out in the distal direction by the pushing shaft to thereby fold back the cover part in the axial direction.

15. The treatment method according to claim 13, comprising:
forming the expansion part by knitting a plurality of elastically deformable wire-like members into a tubular form.

16. The treatment method according to claim 13, comprising:
forming the cover part with at least one hole portion at least on only a distal side of the cover part.

17. The treatment method according to claim 13, wherein the cover part is capable of forming a folded-back part overlapping by being folded back in a circumferential direction such that inner surface portions contact each other.

18. The treatment method according to claim 13, wherein the cover part is configured such that a position at which the cover part contacts the expansion part when contracted and a position at which the cover part contacts the expansion part when expanded are different from each other.

19. The treatment method according to claim 13, wherein at least part of the folded-back part extending in the axial direction when the cover part is contracted is deformable into the overlapping part when the cover part is expanded.

20. The treatment method according to claim 13, further comprising:
a pushing shaft which is a tubular body accommodated in the sheath and penetrated by the shaft part, which is formed to have an inside diameter such that the expansion part and the cover part in the sheath cannot pass therethrough, and which pushes out the expansion part and the cover part from the sheath.
